(19) United States
(12) Patent Application Publication
(10) Pub. No.: US 2016/0074318 A1
(43) Pub. Date: Mar. 17, 2016

(54) Inhalable Pharmaceutical Compositions and the Inhaler Devices Containing Them

(71) Applicant: SANOFI SA, Vernier (CH)

(72) Inventors: Yorick Kamlag, Frankfurt am Main (DE); Mathias Keil, Frankfurt am Main (DE)

(21) Appl. No.: 14/787,727

(22) PCT Filed: Apr. 28, 2014
(86) PCT No.: PCT/EP2014/058616

§ 371 Date: Oct. 28, 2015

(30) Foreign Application Priority Data
Apr. 29, 2013 (EP) 13165799.1

(51) Int. Cl. A61K 9/00; A61K 31/573; A61M 15/00; A61K 31/58; A61K 47/26; A61K 31/137; A61K 31/167

(52) U.S. Cl. CPC A61K 9/008; A61K 31/137; A61K 31/573; A61K 31/167; A61K 31/58; A61K 47/26; A61M 15/0021; A61M 15/0065; A61M 2202/064

(57) ABSTRACT

An inhalable pharmaceutical composition suitable for giving improved feedback to the patient about the inhalation of the metered doses in a range between 5 miligram and 10 miligram and suitable for use in a dry powder inhaler comprises 70 mass %-99.5 mass % of pharmaceutically acceptable carrier suitable for use in dry powder inhalation formulation and 0.5 mass %-30 mass % of one or two pharmaceutically active ingredient(s).

The present invention relates to inhalable pharmaceutical formulations suitable for providing improved feedback to the patient about the inhalation of metered doses and they are the suitable for use in dry powder inhalers. The invention also relates to this dry powder inhaler containing said pharmaceutical formulations. The invention also relates to the method of making these formulations. Furthermore the invention relates to the use of the formulations and the inhalers containing them for the treatment of asthma and COPD.

### SUMMARY OF THE INVENTION

In a first aspect of the invention there is provided certain inhalable pharmaceutical compositions suitable for giving improved feedback to the patient about the inhalation of the metered doses of the formulation and they are suitable for use in dry powder inhalers comprising
at least one pharmaceutically acceptable carrier suitable for use in dry powder inhalation formulations in 70-99.5 mass % and
one or two pharmaceutically active ingredients(APIs) in 0.5-30 mass %.

In the second aspect of the invention there is provided a method of making an inhalable pharmaceutical composition comprising the following steps:

(i) first part of the carrier and first pharmaceutically active ingredient are blended together a mixture is prepared,
(ii) second part of the carrier and optionally the second pharmaceutically active ingredient are blended together a mixture is prepared,
(iii) mixtures received in steps (i) and (ii) are blended together, and
(iv) the received dry powder mixture in step (iv) is filled into the dry powder inhaler.

In the third aspect of invention there is provided a use of the above pharmaceutical compositions in the treatment of asthma or COPD or bronchitis.

In the fourth aspect of the invention there are dry powder inhalers or an assembly of or for an inhaler comprising:
a storage chamber, in which a pharmaceutical composition according to the invention and optionally made by the process of the invention is arranged;
a mouthpiece;
a metering rod provided with a metering chamber, the metering chamber being movable relative to the storage chamber to retrieve a sub-quantity of the pharmaceutical composition from the storage chamber and to move the sub-quantity into a transfer location outside the storage chamber, wherein, in the transfer location, the sub-quantity is arranged to be retrieved from the metering chamber and to be moved to the mouthpiece by a suction airstream which is generatable by a user suction action which is performed for an inhalation, the suction airstream flowing through a flow channel which is establishable between an air inlet of the assembly and the mouthpiece;
a piston which is movable relative to the metering rod between a blocking position, in which the flow channel is blocked by the piston, and a non-blocking position, in which air is permitted to flow from the air inlet to the mouthpiece via the flow channel, the movement of the piston being actuatable by the user suction action—containing at least one pharmaceutically acceptable carrier suitable for use in dry powder inhalation formulations and two pharmaceutically active ingredients.

### DETAILED DESCRIPTION

There are considerable advantages of the pulmonary drug delivery:
It requires a small fraction of delivery of oral dose.
Rapid onset of action.
Low concentration in the systemic circulation which are associated with reduced systemic side effects.
Degradation of drug by liver is avoided by this delivery method.
This delivery is needle-free method.
Gastrointestinal upset may be avoided.

In case of pharmaceuticals the therapeutic window means the range of doses of pharmaceutically active ingredients (APIs) that are actually effective in treating a particular disease. Doses below the therapeutic window are too weak to reach any effect (the blood-level of the API is too low) and doses above window cause unacceptable side-effects (the blood-level of the API is too high).

It is a continuous task to improve/to widen the therapeutic window in case of pharmaceuticals.

In case of inhalable formulations a further simultaneous task to provide such metered doses which gives an improved feedback to the patient about the inhalation of each metered doses.

It is known that the inhalable formulations contain APIs of microgram order of magnitude. The inhalation of such a small amount of solid material is inperceptible. If relative high amount of pharmaceutically acceptable carrier is added to the APIs this may improve the perceptibility of the inhaled dose but it may influence the therapeutic window.

It has been examined how it is possible to improve/widen the therapeutic window and inhalation perceptibility of antiasthmatic API combinations using different inhalable dry powder formulations in inhalators and different methods of their making.

It has been found that if the

(i) first part of the carrier and first API are blended together, a mixture is prepared,
(ii) second part of the carrier and second API are blended together a mixture is prepared,
(iii) mixtures received in steps (i) and (ii) are blended together, and
(iv) the received dry powder mixture in step (iv) is filled into the a dry powder inhaler then the received inhalable pharmaceutical compositions.

Pharmaceutical compositions thus obtained may comprise 70 mass %-99.5 mass % of pharmaceutically acceptable carrier suitable for use in dry powder inhalation formulation and 0.1 mass %-12.5 mass % of a pharmaceutivally active ingredient (API) and 0.4 mass %-17.5 mass % of a second API both of them applicable for treating asthma, COPD or bronchitis.

Possible APIs are among others levalbuterol, reproterol, clenbuterol, bambuterol, broxaterol salmeterol xinafoate, fluticason propionate budesonide, salbutamol, beclomethasone, formoterol, terbutalin, salmeterol, tulobuterol, indacaterol, isoprenaline, hexoprenaline, tiotropium, ipatropium, oxitropium, fenoterol, mometason, ciclesonid or rofleponid.

For the preparation of formulations according to the present invention the pharmaceutically acceptable carriers may be among others lactose, glucose, maltose, fructose, sucrose, dextrose, their hydrates or mixtures thereof. Their particle size distributions may be different.

One group of pharmaceutical compositions thus obtained may comprise 70 mass %-99.5 mass % of pharmaceutically acceptable carrier suitable for use in dry powder inhalation formulation and 0.1 mass %-12.5 mass % of salmeterol xinafoate and 0.4 mass %-17.5 mass % of fluticasone propionate.

Preferred group of pharmaceutical compositions thus obtained may comprise 88 mass %-99.2 mass % of pharmaceutically acceptable carrier suitable for use in dry powder inhalation formulation and 0.3 mass %-5.0 mass % of salmeterol xinafoate and 0.5 mass %-7.0 mass % of fluticasone propionate.

Applied salmeterol xinafoate and fluticasone propionate may have different particle size distributions according to different Pharmacopeias. These APIs having properties as described in Ph. Eur. are well-applicable. Carriers having properties as described in Ph. Eur. are generally applicable.

Particle size distribution may be measured for example by a dry method using laser light scattering in a Helios apparatus. This method is applicable in case of salmeterol xinafoate and fluticasone propionate as well.

Pharmaceutical formulations according to the present inventions may be used for the treatment of asthma, COPD and bronchitis. During the treatment the patient actuates the dry powder inhaler containing the composition of the invention and inhales the pharmaceutical composition.

Due to the pharmaceutically acceptable carrier, the patient furthermore experiences a powdery taste in the mouth upon inhaling the dose of the pharmaceutical composition. This powdery experience of the carrier can for example also provide a significant feedback to the patient about the inhalation of the metered dose.

The pharmaceutical compositions of the invention as described above and optionally made by the process of the invention may be loaded in a dry powder inhaler. The inhaler may be a single dose inhaler or a multiple dose inhaler.

One aspect of this disclosure relates to an assembly for an inhaler. A further aspect of this disclosure relates to an inhaler comprising the assembly. Accordingly, the features disclosed below in connection with the assembly also apply for the inhaler and vice versa.

In an embodiment, the assembly comprises a storage chamber. A powdery substance may be arranged within the storage chamber. The powdery substance may comprise one or more medicinal substances and, preferably, a carrier for the medicinal substance(s).

In an embodiment, the assembly comprises a mouthpiece. The mouthpiece may be provided for interaction with a user. The user may interact with the mouthpiece, e.g. by placing his lips around the mouthpiece, to perform a user suction action for inhalation. The suction action may generate a suction airstream which may flow from an air inlet of the assembly towards the mouthpiece through a flow channel which can be established between the air inlet and the mouthpiece.

In an embodiment, the assembly comprises a metering rod which is provided with a metering chamber. The metering chamber is designed to retain a sub-quantity of the powdery substance. The metering chamber may be a transverse bore extending through the metering rod. The metering chamber is expediently moveable relative to the storage chamber to retrieve the sub-quantity of the powdery substance from the storage chamber and to move the sub-quantity into a transfer location outside the storage chamber. In the transfer location, the sub-quantity may be arranged to be retrieved from the metering chamber and to be moved to the mouthpiece by the suction airstream. The movement from the storage chamber to the transfer location may comprise concurrent axial and rotational movement of the metering chamber with respect to the storage chamber. In this way, a reliable filling of the metering chamber with powder is facilitated. The metering chamber is expediently arranged offset from the axis around which it rotates during its movement from the storage chamber towards the transfer location.

In an embodiment, the assembly comprises a piston. The piston is expediently moveable relative to the metering rod between a blocking position and a non-blocking position. In the blocking position, the flow channel may be blocked by the piston. In the non-blocking position, air is permitted to flow from the air inlet to the mouthpiece via the flow channel. The movement of the piston may expediently be actuated by the user suction action. When the piston is in the blocking position, the piston may cover the metering chamber. Consequently, the sub-quantity of the powdery substance is protected by the piston when it is in the transfer location, e.g. from moisture or from falling out of the metering chamber. As the movement of the piston may be actuated by the user suction action, the piston may be moved only if an inhalation takes place. Hence, the sub-quantity of substance is exposed to the environment and thus to the moisture in the environment only if the piston is moved to uncover the metering chamber and, consequently, only if an inhalation takes place. Further, it is, on account of the piston, guaranteed that no medicament is lost if the metering chamber remains in the transfer location for a longer time or the assembly is subjected to external forces, such as by shaking, for example.

In an embodiment, the assembly comprises a closure cap. The closure cap may be disconnectable from the remainder of the assembly and, preferably, re-connectable to the remainder of the assembly. The closure cap may cover the mouthpiece, particularly if it is connected to the remainder of the assembly. Accordingly, the closure cap may protect the interior of the assembly against exposure to environmental influences such as moisture, for example.

The cap may be releasably connected to the metering rod. Consequently, a movement of the closure cap to uncover the mouthpiece may be converted into movement of the metering rod, such as to move the metering chamber into the transfer location. The connection between the metering rod and the closure cap may be released when the metering chamber has reached the transfer location. Once the metering rod is disconnected from the closure cap, the closure cap may be disconnected from the remainder of the assembly. Therefore, the movement for disconnecting the closure cap from the remainder of the device may be utilized to move the sub-quantity of the powdery substance into the transfer location. No extra user action is required for this purpose.

When the closure cap is re-connected to the remainder of the assembly, the connection between the closure cap and the metering rod may be re-established. Also, when the closure cap is reconnected to the remainder of the assembly, the metering chamber may be moved again from the transfer location into the storage chamber. When the connection between the metering rod and the closure cap is re-established, a preferably audible and/or tactile feedback may be generated. In this way, it may be indicated that the closure cap has been re-connected correctly and that the assembly is ready to move the metering chamber again from the storage chamber into the transfer location. Thus, the feedback provides information to the user that the assembly may be used again for performing a further inhalation. An audible feedback may be generated by a snap-fit engagement which is re-established between the closure cap and the metering rod. Again, for moving the metering chamber back into the storage chamber, no extra user action is necessary besides the re-connection of the closure cap to the remainder of the assembly.

In an embodiment, the piston is retained in the non-blocking position after the user suction action. The piston may be retained in this position at least until the closure cap is being reconnected to the remainder of the assembly. During re-connection of the closure cap to the remainder of the assembly, the piston may be moved from the non-blocking position back to the blocking position. For doing so, no extra user action is necessary besides the re-connection of the closure cap to the remainder of the assembly.

In an embodiment, the assembly comprises a window which allows a visual check to confirm whether the assembly is in a ready-to-inhale state or not. For example, if the assembly is in the ready-to-inhale state, the piston, which is in the blocking position, may be visible through the window. When the closure cap is connected to the remainder of the assembly, the window is expediently covered by the cap and is preferably not visible to the user. For example, the piston may be green. The presence of the piston in the visible area defined by the window signals to the user that the assembly is ready for an inhalation. If the piston is not visible through the window, the window may be black, for example. If the piston is not visible, it may be in the non-blocking position, which is the position after inhalation and before closing the device again by re-connecting the closure cap to the remainder of the assembly.

In an embodiment, the assembly is configured to provide one or more operational feedbacks, such as audible, visual, tactile and/or inspiratory feedback, to the user. The respective feedback may indicate to the user that the inhalation is being performed correctly or has been performed correctly. Preferably, a plurality of feedbacks is provided to the user. The first of these feedbacks may be the visual check whether the assembly is in the ready-to-inhale state. The second one may be a feedback which is provided when the piston begins to move from the blocking position to the non-blocking position. This feedback may be an inspiratory feedback as the inspiratory effort required to get the piston moving is greater than the one necessary to keep the piston moving. Accordingly, once the inspiratory effort is reduced, a feedback is given to the user which indicates that the piston has moved. Once the piston reaches the blocking position, an engagement of the piston with an end stop may provide a further feedback, particularly an audible one, to the user that the piston has reached the blocking position. Expediently, the inspiratory effort necessary to move the piston is chosen such that it is guaranteed that the whole sub-quantity of powdery substance retained in the metering chamber is transferred by the suction airstream, which is generated by the user suction action, to the user and is deposited in the user's lung. The inspiratory effort to move the piston may be adjusted by adjusting the static friction which has to be overcome to move the piston.

In an embodiment, the assembly comprises a housing. The storage chamber may be arranged within the housing. The closure cap may be releasably connected to the housing when it is connected to the remainder of the assembly. Releasing the connection or re-establishing the connection to the housing may require a rotational and preferably a concurrent axial movement of the closure cap with respect to the housing. The closure cap, when connected to the housing, may be threadedly engaged with the housing.

In an embodiment, the metering rod is rotated and moved axially with respect to the housing when the metering chamber is moved from the storage chamber into the transfer location.

In an embodiment, the assembly comprises an outer part, such as an outer cylinder, for example. The mouthpiece may be provided on the outer part. The outer part may be uncovered once the closure cap has been disconnected from the remainder of the assembly. The outer part may be connected rotatably to the housing. Thus, relative rotation between housing and outer part may be allowed. The outer part may be axially secured with respect to the housing. Thus, relative axial movement between housing and outer part may be prevented or restricted. The closure cap, when connected to the housing, may be rotationally locked to the outer part such that the outer part rotates together with the closure cap when the closure cap is disconnected from the housing. The metering rod may be retained in the outer part. The metering rod may rotate when the outer part rotates. Accordingly, when the cap is disconnected, the metering chamber may rotate within the storage chamber, thereby being filled with the sub-quantity of the powdery substance.

In an embodiment, the mouthpiece is coloured differently than a region of the assembly which adjoins the mouthpiece, e.g. the outer part. Consequently, the outer part may be coloured differently than the mouthpiece. In this way, the user knows where to place his/her lips. As the outer part may be provided with one or more additional openings, such as the air inlet, for example, it is important to ensure that the suction action is applied via the mouthpiece only and that the user does not attempt to use a different opening for this purpose.

In an embodiment, the metering rod extends through the piston, with the piston being preferably slidable along the metering rod. The piston may be in frictional contact with the metering rod or not.

In an embodiment, the storage chamber is sealed with respect to the environment. Particularly, the metering rod may comprise a region outside the metering chamber, which is always arranged within the storage chamber. In this way, a guiding opening to the storage chamber may receive the metering rod and always be filled with a part of the metering rod, regardless of whether the metering chamber is arranged in the transfer location or in the storage chamber. Consequently, the guiding opening may be sealed by means of the metering rod and, if applicable, by means of a seal provided in the region of the opening and frictionally cooperating with the metering rod to seal the storage chamber. Accordingly, the powdery substance within the storage chamber may be protected against harmful environmental influences like moisture, for example.

In an embodiment, the assembly comprises a first rotational stop feature on the closure cap, the first rotational stop feature providing a first rotational stop face. Further, the assembly may comprise a second rotational stop feature on the remainder of the device, preferably on the housing, the second rotational stop feature providing a second rotational stop face. The first and the second stop faces may extend in an axial direction. The first and the second stop faces may face in the angular direction. The first and the second stop faces are expediently designed to abut each other when the closure cap has reached its end position when being reconnected to the remainder of the assembly. Thus, the relative arrangement of the first and second stop features may indicate whether the closure cap has reached its end position. The first and second rotational stop features are preferably arranged to be visible when the closure cap is (re-)connected to the remainder of the assembly, particularly also when the first and the second stop faces abut. In this way, the user can perform a simple visual check to confirm that a defined end position of the closure cap has been reached. As the closure cap plays a vital role in preparing the assembly for use, this check increases the user confidence in the reliability of the device.

When the closure cap is connected to the housing by means of rotational and preferably concurrent axial movement, the first stop face may be moved towards the second stop face, e.g. along a helical path defined by the threaded connection between housing and closure cap, until the first and second stop faces abut. If the two stop faces abut, no further rotation is possible. Accordingly, this is advantageous as compared to two stop faces which face in the axial direction, for if those stop faces abut, a user might try to rotate the cap further, thereby deforming and destroying the cap. If two angular stop faces abut, the risk of destroying the cap can be considerably reduced.

In an embodiment, the assembly comprises an indicator which provides information about the amount of powdery substance remaining in the storage chamber. The indicated information may be information directly derived from the filling level of the storage chamber. For example, the assembly may comprise an indicator window displaying an indicator that is directly connected to an element which delimits the storage chamber. The indicator window may be visible when the closure cap is connected to the remainder of the assembly. The element which delimits the storage chamber may, for example, be a moveable wall of the storage chamber, which may be spring-biased. Once one or a plurality of sub-quantities of the powdery substance has been removed from the storage chamber, the moveable wall may be moved to keep the powdery substance within the storage chamber compact. The indicator may be moved within the window concurrently with the moveable wall. The position of the indicator within the window may indicate the filling level of the storage chamber.

According to one embodiment, herein, particularly with respect to the composition or the assembly, the term “D50 less than 3-6 micrometer” means that the D50 value is less than 6 micrometer, or less than 3 micrometer. In another embodiment, the term “D50 less than 3-6 micrometer” means that the D50 value is between 3 and 6 micrometer.

In an advantageous embodiment of invention an assembly of an inhaler comprising:
a storage chamber, in which a pharmaceutical composition according to the invention and optionally made by the process of the invention is arranged;
a mouthpiece;
a metering rod provided with a metering chamber, the metering chamber being movable relative to the storage chamber to retrieve a sub-quantity of the pharmaceutical composition from the storage chamber and to move the sub-quantity into a transfer location outside the storage chamber, wherein, in the transfer location, the sub-quantity is arranged to be retrieved from the metering chamber and to be moved to the mouthpiece by a suction airstream which is generatable by a user suction action which is performed for an inhalation, the suction airstream flowing through a flow channel which is establishable between an air inlet of the assembly and the mouthpiece;
a piston which is movable relative to the metering rod between a blocking position, in which the flow channel is blocked by the piston, and a non-blocking position, in which air is permitted to flow from the air inlet to the mouthpiece via the flow channel, the movement of the piston being actuatable by the user suction action. The advantages of this embodiment become apparent from the description above and the description of the exemplary embodiments below.

Further features and expediencies of the inhaler disclosure become apparent from the following description of the exemplary embodiment in conjunction with the figures.

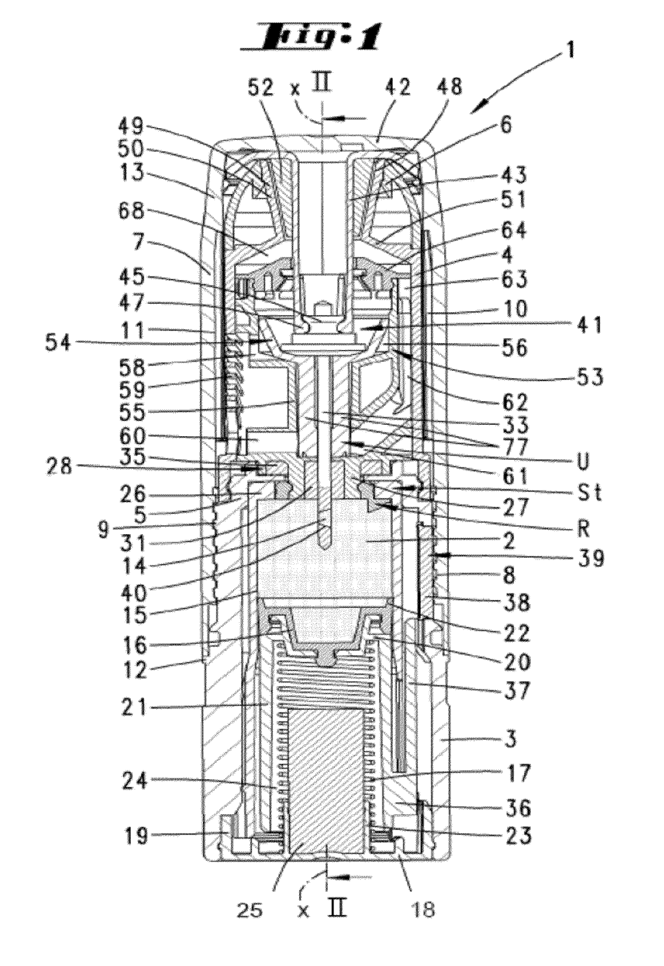

FIG. 1 shows the vertical section through a metering device according to the invention in its basic position, with the cap closed;

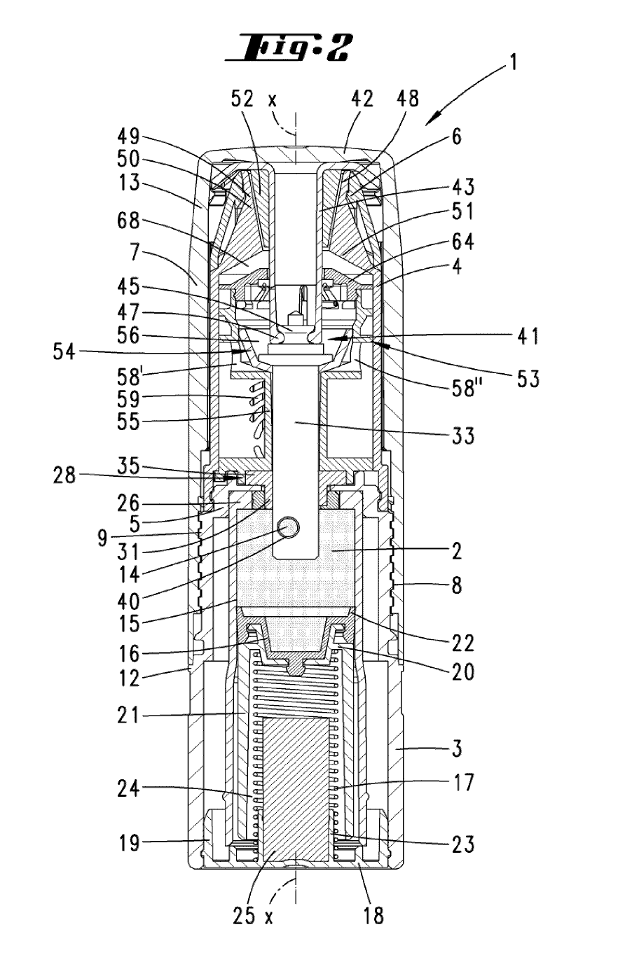

FIG. 2 shows a further vertical section along line II-II in FIG. 1;

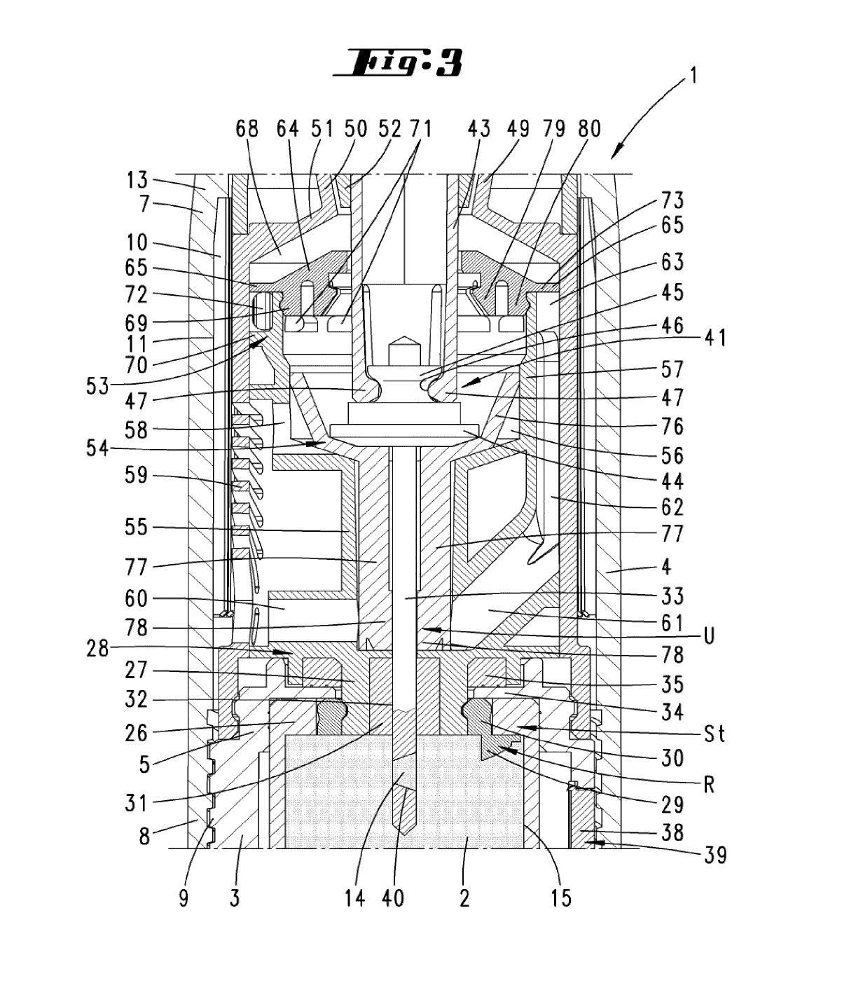

FIG. 3 shows an enlargement of an upper region of the device according to FIG. 1;

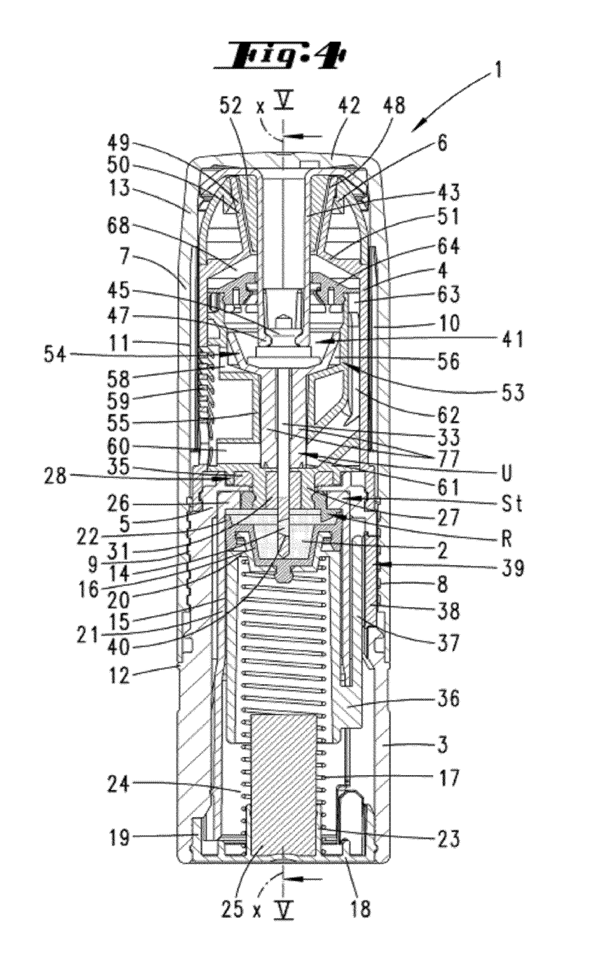

FIG. 4 shows a sectional illustration corresponding to FIG. 1, relating to the situation where the storage chamber for the substance which is to be inhaled has been more or less emptied;

FIG. 5 shows the section along line V-V in FIG. 4;

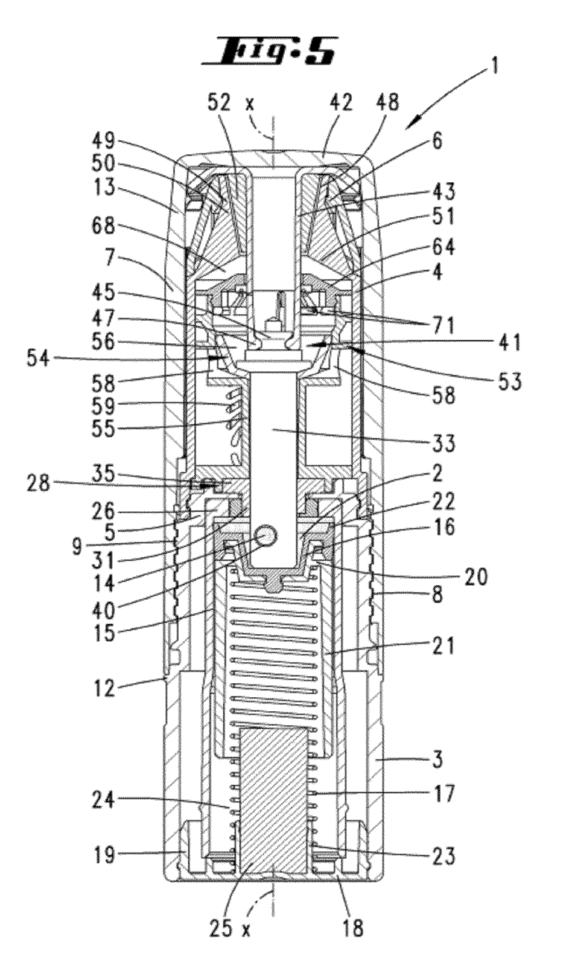

FIG. 6 shows a further illustration corresponding to FIG. 1, this time during removal of the closure cap;

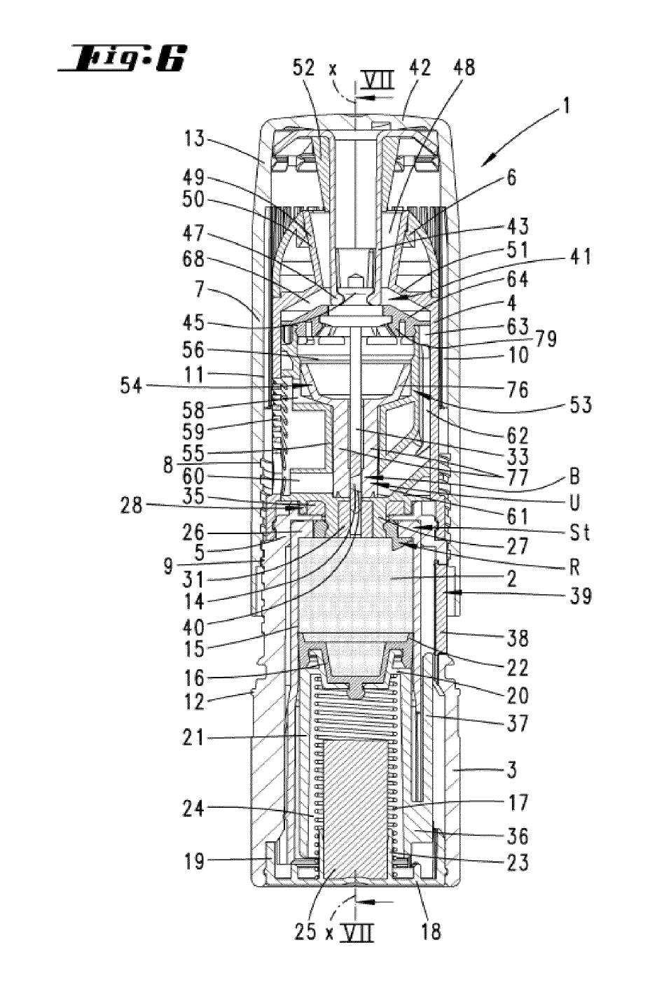

FIG. 7 shows the section along line VII-VII in FIG. 6;

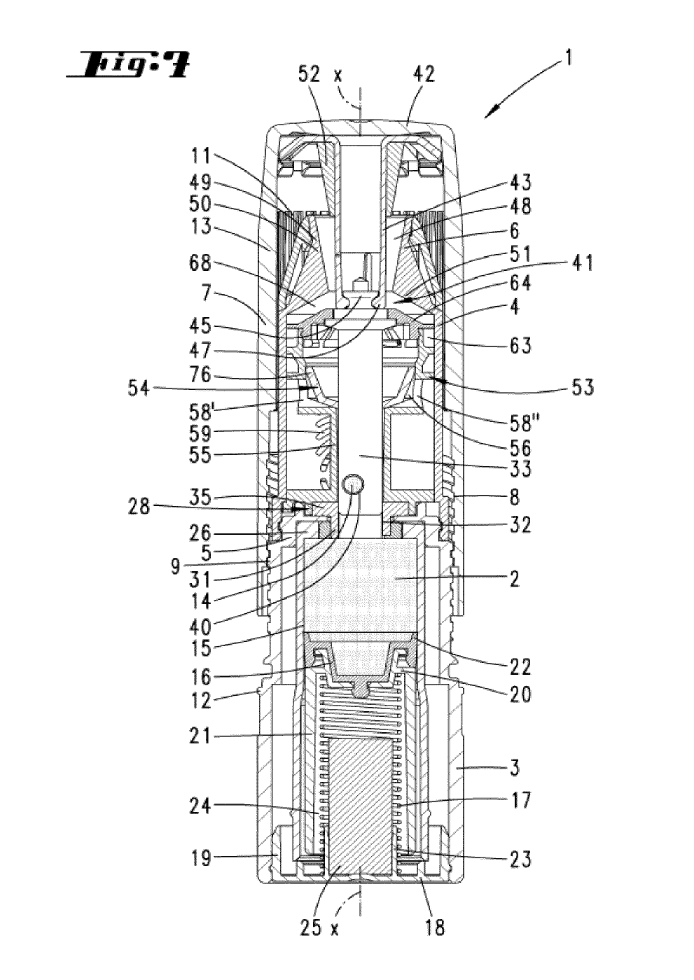

FIG. 8 shows the vertical section according to FIG. 1, but following removal of the closure cap and the resulting displacement of a metering chamber into the emptying-standby position;

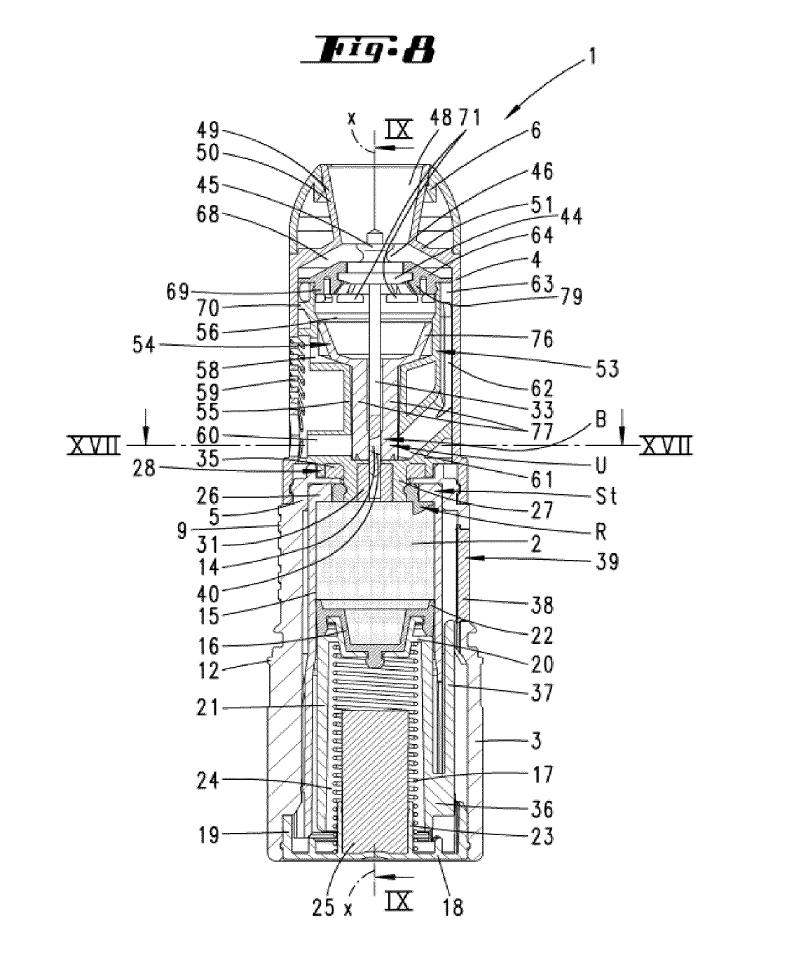

FIG. 9 shows the section along line IX-IX in FIG. 8;

FIG. 10 shows a detail-view illustration corresponding to FIG. 3, relating to the situation according to FIG. 8;

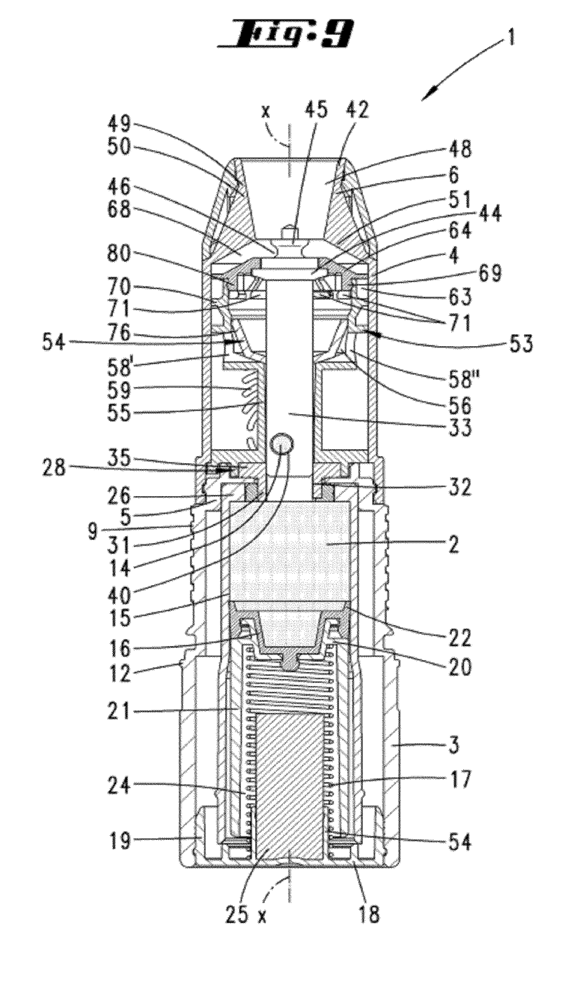

FIG. 11 shows a follow-up illustration to FIG. 8, but relating to a position assumed during inhalation;

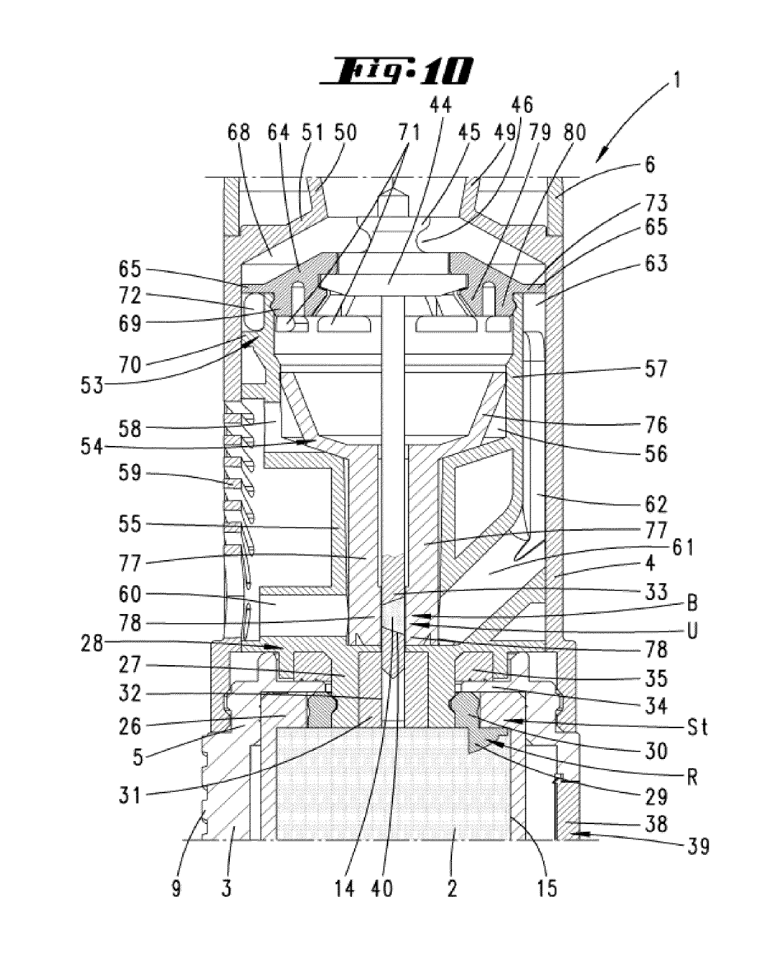

FIG. 12 shows the section along line XII-XII in FIG. 11;

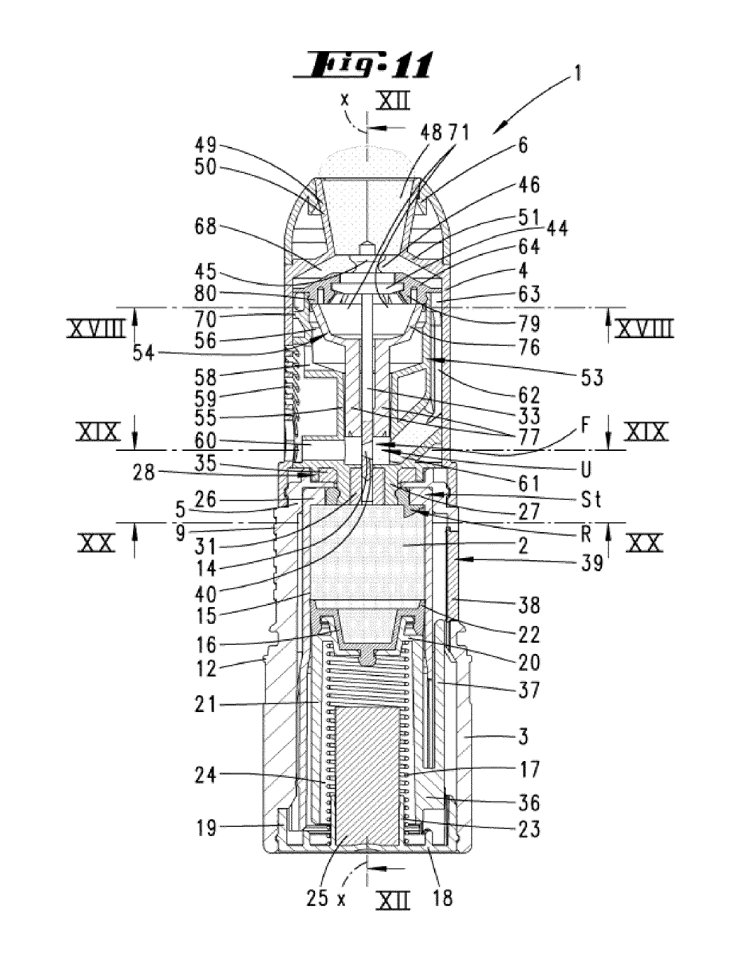

FIG. 13 shows a further detail-view illustration corresponding to FIG. 3, but relating to the situation according to FIG. 11;

FIG. 14 shows a further vertical-section illustration corresponding to FIG. 1, this time relating to an intermediate position as the closure cap is being replaced following completion of inhalation;

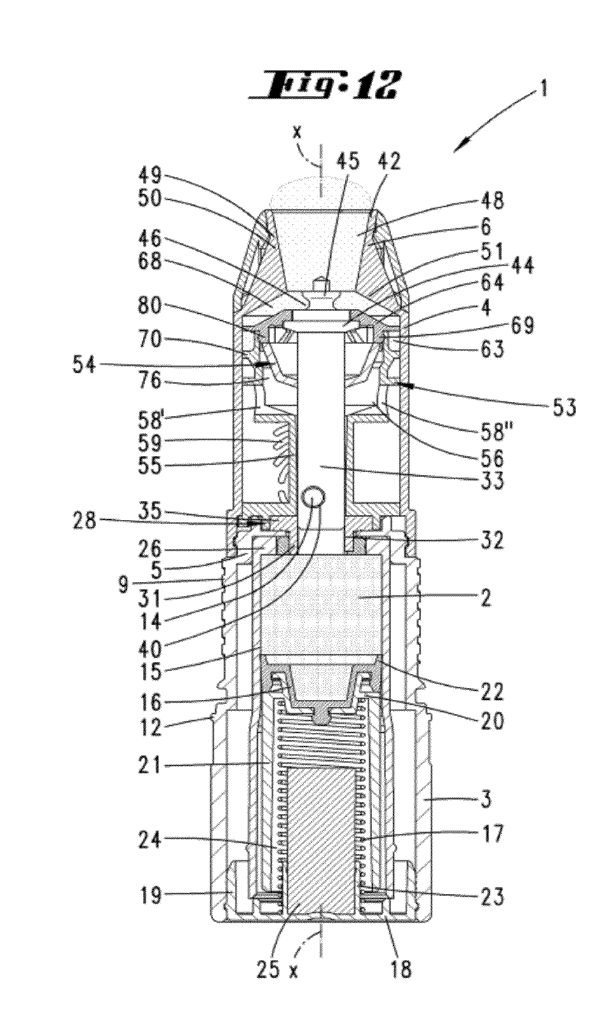

FIG. 15 shows a follow-up illustration to FIG. 14, relating to an intermediate position;

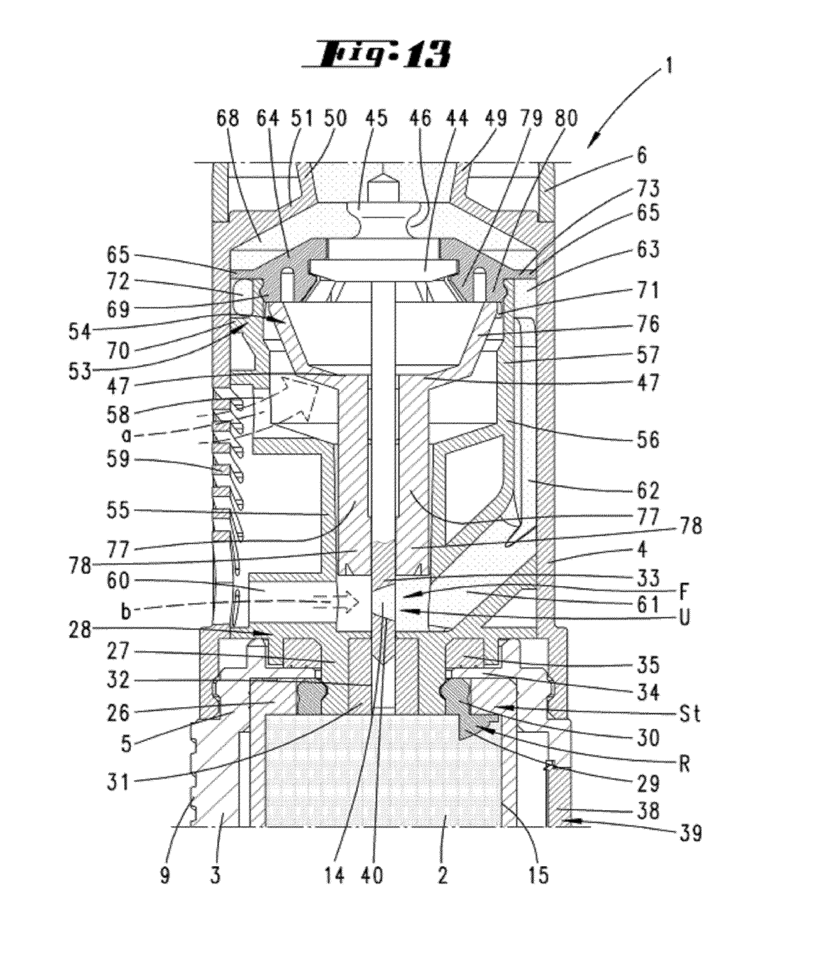

FIG. 16 shows a follow-up illustration to FIG. 15, relating to an intermediate position as the operation of screwing on the closure cap continues;

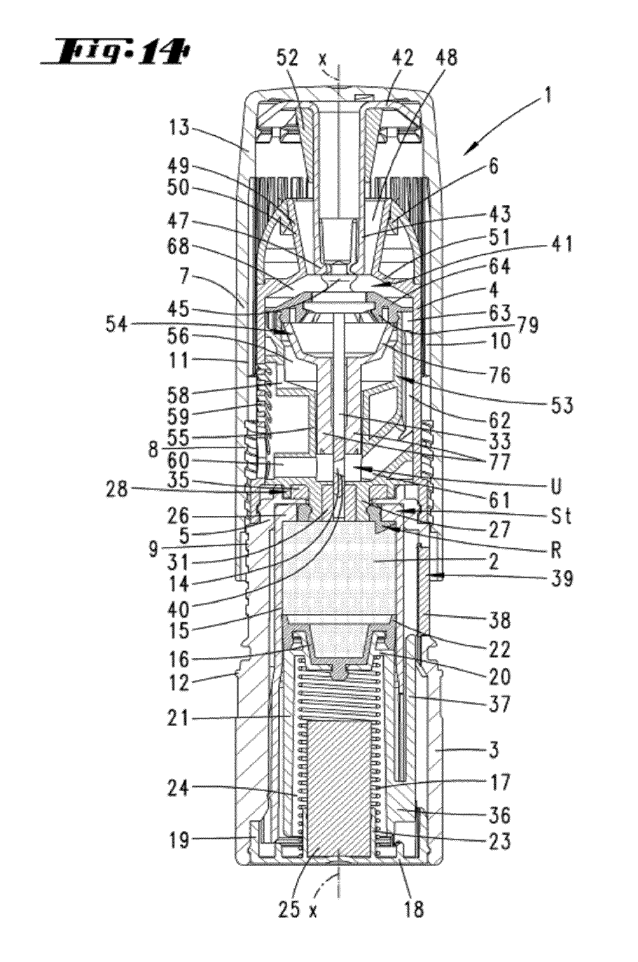

FIG. 17 shows the cross-section through the metering device in the emptying-standby position along line XVII-XVII in FIG. 8;

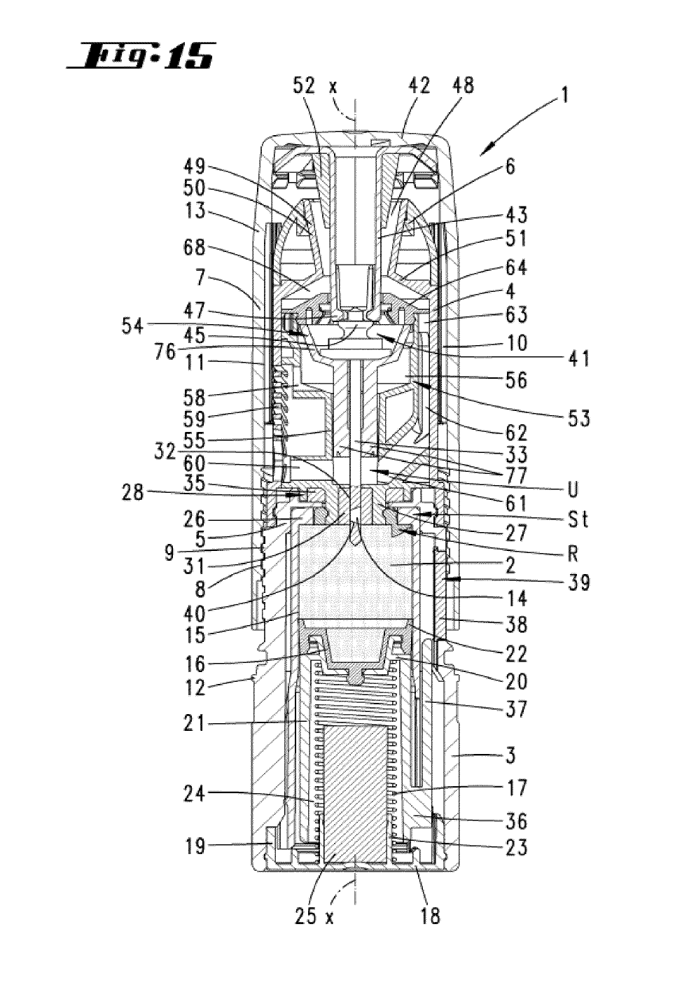

FIG. 18 shows the cross-sectional illustration through the metering device along line XVIII-XVIII in FIG. 11;

FIG. 19 shows an illustration which corresponds to FIG. 17 and has been taken along line XIX-XIX in FIG. 11, relating to the emptying-release position;

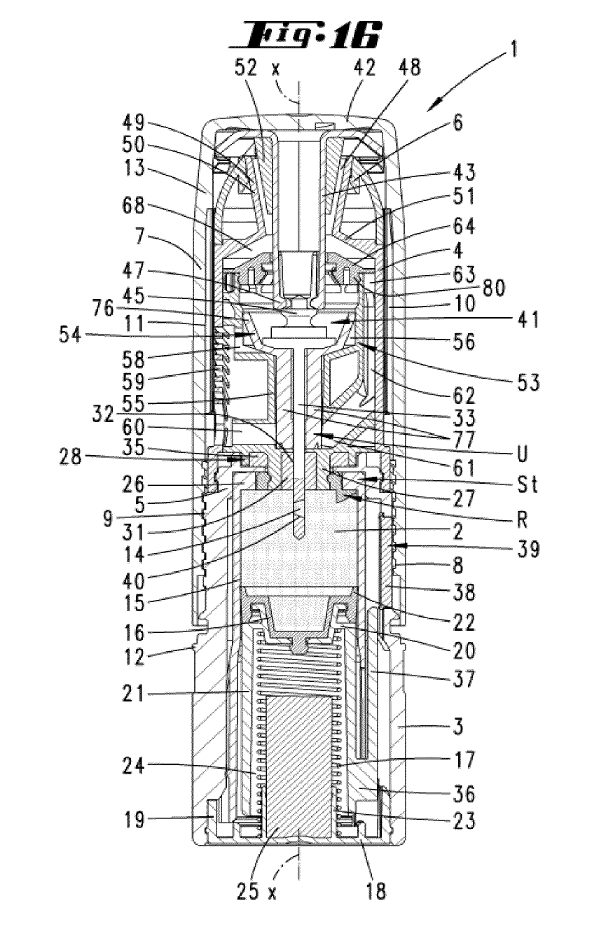

FIG. 20 shows the section along line XX-XX in FIG. 11 through the storage chamber, with the substance which is stored here having been left out;

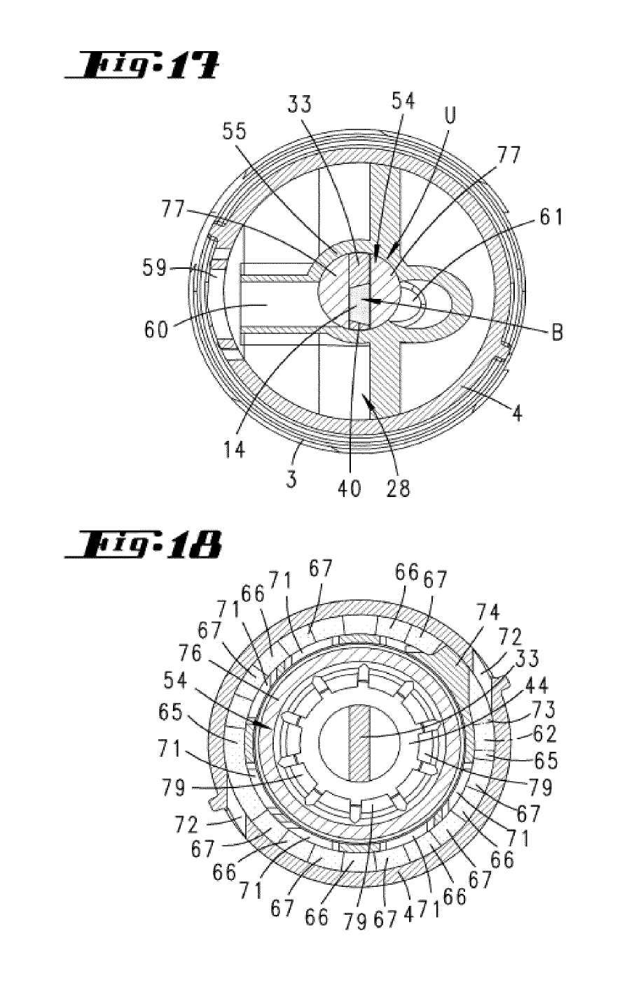

FIG. 21 shows a perspective detail illustration of an inner cylinder of the metering device;

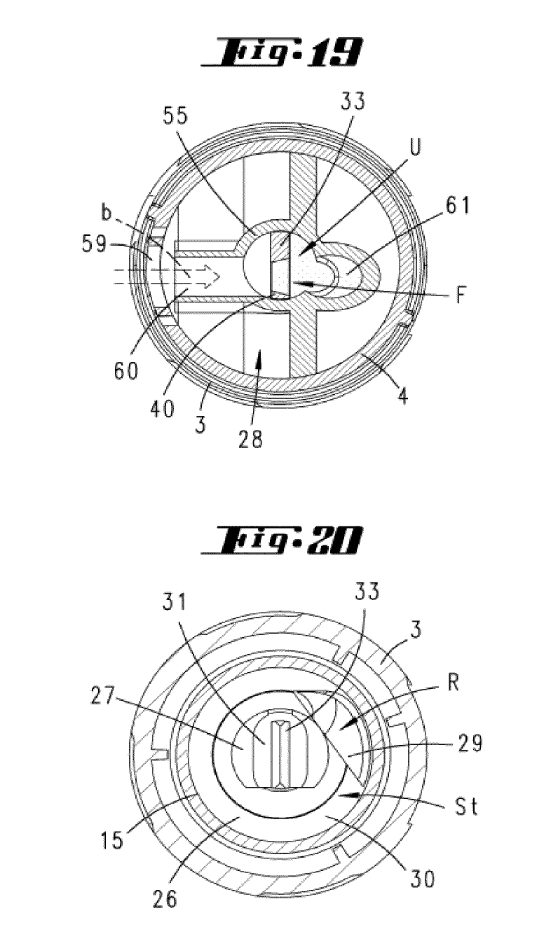

FIG. 22 shows a further perspective illustration of the inner cylinder;

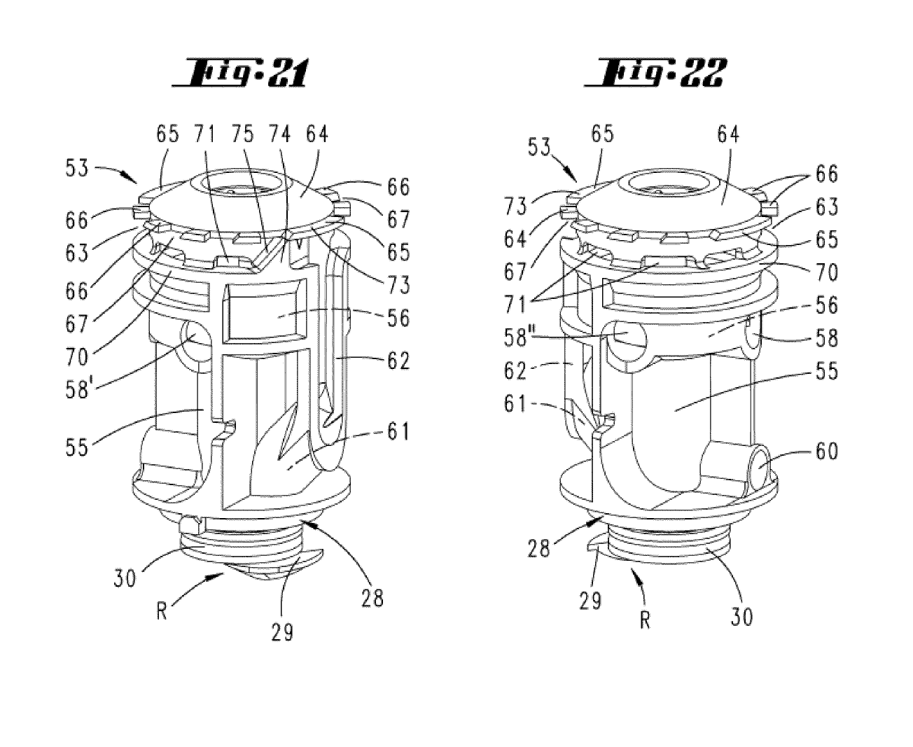

FIG. 23 shows a perspective detail illustration of the metering rod of the metering device;

FIG. 24 shows a perspective detail illustration of the piston;

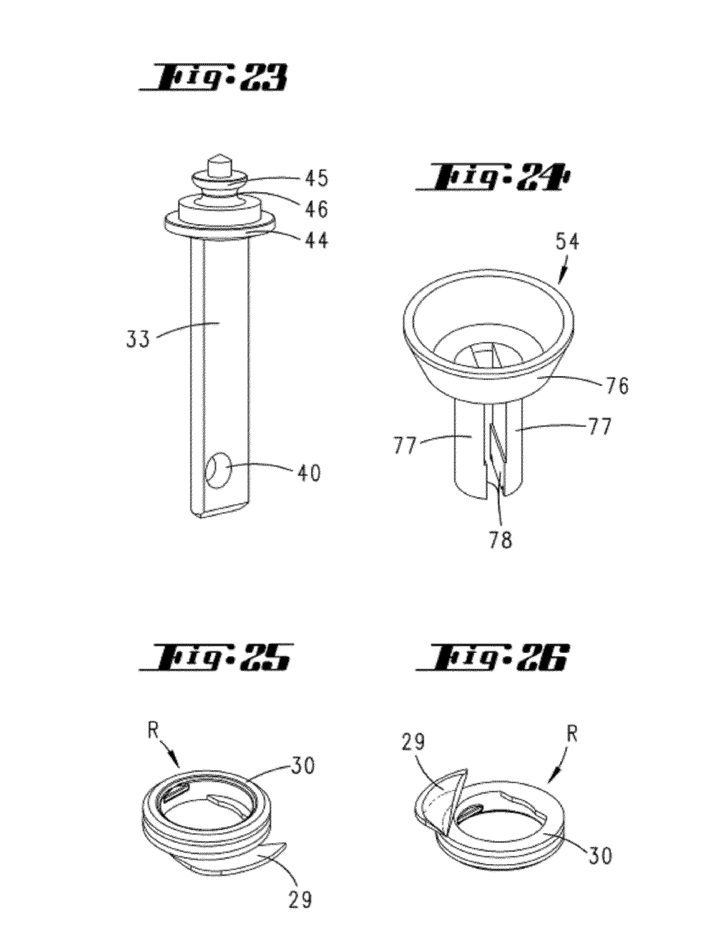

FIG. 25 shows a further perspective detail illustration of a rotor-like blade for disposing on the inner cylinder;

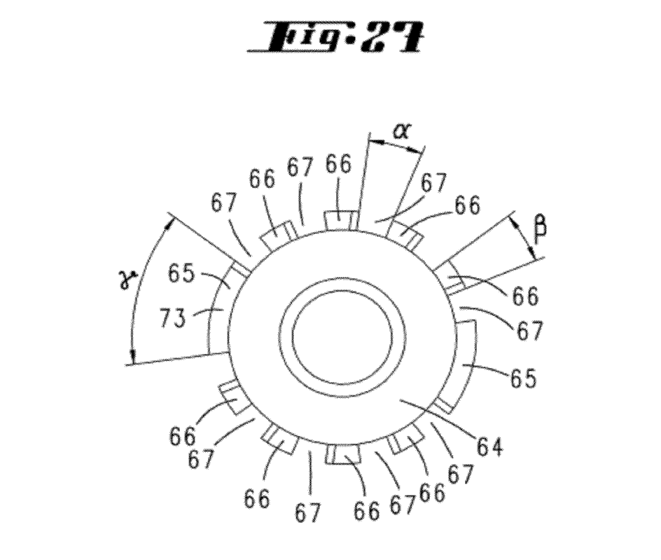

FIG. 26 shows a further perspective illustration of the rotor-like blade;

FIG. 27 shows, in a detail drawing, the bottom view of a cover of an annular chamber; and

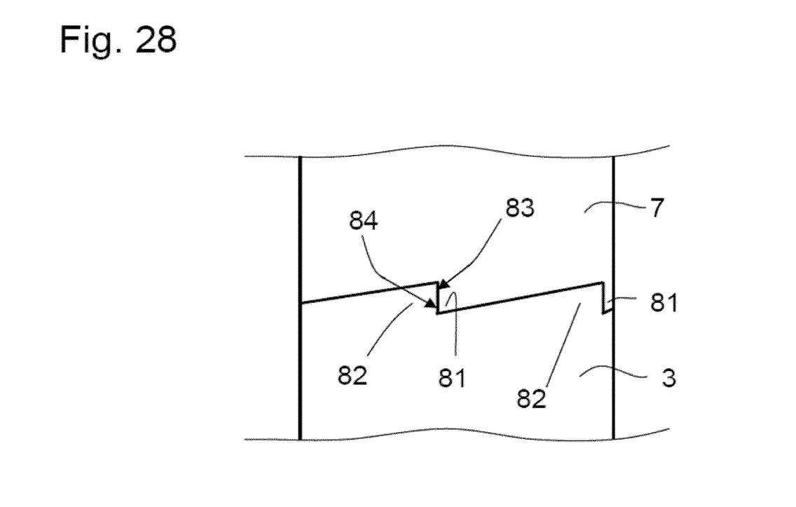

FIG. 28 shows a schematic view of a part of the metering device of the previous figures in the closed state.

Like elements, elements of the same kind and identically acting elements may be provided with the same reference numerals in the figures.

The metering device 1, e.g. an inhaler, which is illustrated in the figures and is intended for the inhalation of a powdery or pulverulent substance 2, in particular a medicinal substance, is preferably realized as a short-elongate device which can readily be carried in a pocket. The metering device 1 has a, preferably cylindrical, housing 3 which determines its shape.

The, for example cylindrical, tube-like, housing 3 has, at its head end, an outer part or outer cylinder 4 which can be rotated about the device axis x relative to the housing 3.

This outer cylinder is secured in a rotatable manner on the housing 3, such as in the region of an end-side radial step 5.

This likewise cylindrical, tube-like outer cylinder 4 merges, at the head end of the device 1, into an attached mouthpiece 6, which is formed appropriately for the mouth, i.e. which is, for example, flattened. This mouthpiece 6 can have a cup-like closure cap 7 engaging over it in a protective manner. This closure cap is realized as a screw cap, for the reason of which an associated internal thread 8 engages with a corresponding external thread 9 on the lateral wall of the housing 2.

The outer cylinder 4 is connected to the closure cap 7 in a rotationally fixed manner, for the reason of which the outer cylinder has, on the outside of its lateral wall, vertically oriented ribs 10 which interact with correspondingly positioned, slot-like vertical grooves 11 on the inside of the wall of the closure cap 7. Accordingly, screw-action actuation of the closure cap 7 causes the outer cylinder 4 to be rotated about the device axis x.

At the foot end, the end periphery of the cup-like closure cap 7 engages in a stop-limiting manner, and with sealing via a cone, against an annular shoulder 12, which is achieved on account of the above-mentioned step of the cylindrical housing 3.

The closure cap 7 serves, at the same time, as an actuating handle 13 for dispensing the pulverulent substance 2 in reproducible sub-quantities 14, for the purpose of which use is made of the axial screw-action displacement provided by the threaded engagement between the internal thread 8 and the external thread 9. The substance 2, e.g. comprising a powder, is accommodated (possibly such that it can be refilled) in a storage chamber 15 of the housing 3. The substance 2 may comprise or consist of the pharmaceutical composition described in more detail above or below. A metering device conveys a respective sub-quantity 14 of substance to a transfer location U located outside the storage chamber 15. For this purpose, a metering rod 33 may be movable such that a metering chamber 40 of the metering rod receives a sub-quantity of the substance 2 within the storage chamber 15, the sub-quantity being transported out of the storage chamber by relative movement of the metering chamber with respect to the storage chamber. The sub-quantity may be moved into the transfer location U by means of the metering chamber.

The meterable substance is a (usually medicinal) pulverulent substance 2. It is possible for basic bodies such as lactose, which are capable for example of transporting a suction stream, to be carriers for fine micronized drug particles adhering to their surface.

The storage chamber 15 is terminated at the bottom by a cup-like pressure-exerting base 16, which is spring-loaded in the direction of the mouthpiece 6 by means of a compression spring 17. The base 16 thus forms a moveable wall of the storage chamber 15. The compression spring 17 has its foot-side end turn supported on a base cap 18, which closes the housing 3 there. This base cap is in latching engagement with that portion of the housing 3 which is of larger cross-section here on its inside wall, a corresponding latching collar 19 of the base cap 18 engaging in a matching annular groove of the housing 3.

The head-side end turn of the biased compression spring 14 subjects an inner shoulder 20 of a hollow piston 21 of the piston-like means 16/21 to loading action. As can be seen from the illustrations, the pressure-exerting base 16, which is in the form of a graduated cup, is connected by means of latching action to the hollow piston 21 in the region of the inner shoulder 20.

The cup periphery of the pressure-exerting base 16 forms an annular lip 22 which, on account of its elastomeric material, strips substance off the wall of the storage chamber 15 without leaving any residues.

In the exemplary embodiment illustrated, the compression spring 17 is a cylindrical spring which, in its relaxed state, has a length corresponding approximately to ten times the maximum contact-pressure length. The contact-pressure length is defined by the extent of axial displacement of the pressure-exerting base 16 between a lower position according to FIG. 1, this position corresponding to the filling position, and an upper, stop-limited position of the pressure-exerting base 16 in the storage chamber 15 according to FIG. 4. Thus, the exemplary embodiment illustrated provides a contact-pressure length of 15 mm. As a result of the configuration of the spring, in particular as a result of the selected length of the spring, the pressure-exerting base 16 is subjected to a constant spring pressure over the entire contact-pressure length, which causes the substance to be compressed uniformly throughout the duration of use of the device 1.

A hollow upright stub 23 extends centrally from the base cap 18. Together with the hollow piston 21, which encloses it at a distance apart, this hollow upright stub forms a chamber 24 for the compression spring 17. The hollow upright stub 23 contains, in its centre, a moisture-absorbing material in the form of a drying-agent capsule 25. At the transition to the outer cylinder 4, which follows the housing 3 in the axial direction, the storage chamber 15 terminates with a chamber ceiling 26 formed integrally with the lateral wall of the storage chamber 15. Passing through the centre of this chamber ceiling is a cylinder portion 27 of a rotary part 28 which extends in a plane perpendicular to the device axis x.

This rotary part is of substantially plate-like configuration and is connected in a rotationally fixed manner to the outer cylinder 4 and, accordingly, can be rotated about the device axis x in relation to the chamber ceiling 26. The cylinder portion 27 extends on the underside of the rotary part 28, passing through the chamber ceiling 26. The lower free end surface of the cylinder portion 27 is located in the plane of that surface of the chamber ceiling 26 which covers the storage chamber 15.

The diameter of the through-opening in the chamber ceiling 26 is larger than the diameter of the cylinder portion 27. A holder, of annular shape in plan view, for a rotor blade R, is positioned in the annular gap which remains. This rotor blade is connected in a rotationally fixed manner to the cylinder portion 27.

The inner surface of the rotor ring 30, this inner surface being directed toward the storage chamber 15, is located in the plane of the correspondingly directed end surface of the cylinder portion 27.

The rotor R, which is illustrated on its own in FIGS. 22 and 23, carries on its underside, that is to say in the direction toward the storage chamber 15, a blade 29. This is a blade 29 which is in the form of a spherical-cap portion and projects radially outward beyond the ring 30 of the rotor R. The blade 29 correspondingly engages beneath that region of the chamber ceiling 26 which adjoins the rotor R radially on the outside, wherein that surface of the blade 29 which is directed toward the chamber ceiling 26 is of planar configuration. This surface of the blade 29 engages with the top surface of the chamber which is directed toward the blade. The blade 29 extends radially as far as the inner wall of the storage chamber 15. From this radial outer region, the blade 29 slopes up convexly in the radial inward direction, as seen in the cross-section, to an axial height corresponding approximately to the extent by which the blade 29 projects radially beyond the rotor ring 30.

As a result of this arrangement, the blade 29 of the rotor R projects into the substance stored in the storage chamber 15. The shoulder formed by the chamber ceiling 26, in interaction with the blade 29 or rotor R, which can be rotated relative to the storage chamber 15, forms a stator St.

The rotor R is clipped on the cylinder portion 27 of the rotary part 28 via the rotor ring 30.

The cylinder portion 27 accommodates a sealing bushing 31 at its centre. This bushing consists of a rubber material or a similar elastic material. This leaves, at its centre, a cross-sectionally slot-like guide opening 32 for the cross-sectionally adapted metering rod 33.

In the simplest configuration, the sealing bushing 31 and also an annular seal 35 provided between the rotary part 28 and a housing portion 34, which engages over the chamber ceiling 26 on the housing side, may be produced by two-component injection molding together with the rotary part 28 and, furthermore, with an inner cylinder, which will be described in more detail. It is, however, also possible in this respect for the rubber or elastomer parts to be provided subsequently during production.

At the foot end, the hollow piston 21, which is connected by means of latching action to the pressure-exerting base 16, has a radial extension arm 36. Integrally formed on the latter is an axially oriented indicating protrusion 37, which engages over the storage-chamber wall on its outside. The axial position of this indicating protrusion, this position being reached in dependence on the position of the pressure-exerting base, can be seen by the user from the outside through a viewing window 38 provided in the housing. A filling-level indicator 39 is provided as a result.

The metering rod 33 is appropriately configured for functioning as a moving metering chamber 40 for the sub-quantity 14 of substance which is to be dispensed, the metering rod 33 moving linearly along the longitudinal centre axis x-x of the substantially rotationally symmetrical device 1, and this being accompanied by a rotary movement executed about the longitudinal centre axis x-x. The metering rod 33 is formed substantially as a flat part with an elongate-rectangular cross-section. The length ratio of the short side to the long side is approximately 1:3 in the exemplary embodiment illustrated.

At the end which is directed away from the mouthpiece 6, the metering rod 33 forms a portion which tapers more or less in the manner of a cross-recessed screwdriver tip. Here, the two mirror-symmetrical oblique flanks extend from the respective broad sides of the metering rod 33 (cf. FIG. 20).

On account of the metering rod 33 being carried along in rotation, the cross-sectional configuration of the metering rod 33 and the tapering of the free end region have a loosening, displacing effect in the central region in relation to the mass of pulverulent substance 2.

The metering chamber 40 is realized as a transverse hole which runs substantially perpendicular to the longitudinal centre axis x-x and has an axis which passes through the broad-side surfaces of the metering rod 33. The transverse hole is formed conically, so that the transverse hole tapers in the direction of one broad-side surface of the metering rod 33. Furthermore, as can be seen for example from the illustration in FIG. 2, the metering chamber 40, which is formed in the region of that end of the metering rod 33 which projects into the mass of substance, is disposed eccentrically in relation to the broad-side surfaces of the metering rod 33, that is to say it is offset laterally in relation to the longitudinal axis x-x.

The displacement path of the metering chamber 40, which moves linearly and, at the same time, in rotation, allows, in both end positions of the metering rod 33, for the cross-section of the guide opening 32 to be kept closed, with metering-chamber-filling scraping or stripping action over the length of said opening 33.

The mouthpiece end of the closure cap 7 forms a docking location 41 between the metering rod 33 and the closure cap 7, this docking location disengaging when subjected to overloading. The latching means on the closure-cap side here is a resilient hook annulus which is formed in the region of the free end of a hollow cylinder 43 disposed centrally on the underside of a closure-cap ceiling 42. The corresponding end of the metering rod 33 is rotationally symmetrical in cross-section, a disk-like radial collar 44, furthermore, projecting out in the transition region from the flat-part portion to the cylindrical end portion. At an axial spacing from this radial collar 44, that end region of the metering rod 33 which is directed away from the flat part forms a latching head 45. A wasp-waist-like annular groove 46 is formed between this latching head and the radial collar 44. Inwardly directed noses 47 of the resilient tongues of the hook annulus engage in this annular groove. The noses 47 can pass over the latching head 45 in both axial directions. The latching action may be fairly tight, since it is released and reinstated during the screwing-action displacement of the cap.

The central opening 48 of the mouthpiece 6 is formed in the region of a dispersing part 49. This dispersing part 49 opens conically outward, that is to say in the direction away from the storage chamber 15, the wall 50 of the dispersing part merging, in the direction toward the storage chamber 15, into an annular, roof-like ceiling portion 51. At the same time, the latter forms the upper end of the outer cylinder 4, which carries the mouthpiece 6.

The central free space created by the dispersing part 49 has the hollow cylinder 43, which carries the noses 47, passing through it centrally in the cap-closed position. The annular space which is formed here between the hollow cylinder 43 and the dispersing-part wall is filled by a further drying-agent capsule 52 in the cap-closed position.

The outer part or outer cylinder 4 accommodates an inner part or inner cylinder 53, passing through the centre of which is the metering rod 33 and, in the cap-closed position, the hollow cylinder 43 belonging to the closure cap. The inner cylinder is connected in a rotationally fixed manner to the outer cylinder 4.

This inner cylinder 53 is configured substantially as a hollow body and carries, in its centre, an axially displaceable piston 54. The piston 54 is guided more or less in the lower half of the inner cylinder 53, directed toward the storage chamber 15, by a cross-sectionally round guide portion 55.

That portion of the inner cylinder 53 which is directed away from the storage chamber 15 forms a piston-head displacement region 56 which has a cross-section larger than that of the guide portion 55 and the axially oriented wall 57 of which has radial openings 58, 58′ and 58″. These radial openings are in flow connection with a grille-wall portion 59 of the outer cylinder.

Formed beneath the grille-wall portion 59, and furthermore at the foot end of the guide portion 55 of the inner cylinder, is a radially oriented flow channel 60, which likewise opens toward the grille-wall portion 59. This flow channel may also serve as a window for visually monitoring the metering rod 33. It opens out into the free space left in the centre by the guide portion 55. Radially opposite the flow channel 60, the guide portion 55 is adjoined by an intermediate channel portion 61 which, starting from the guide portion 55 and with the inclusion of an angle of 45° in relation to a plane oriented perpendicularly to the axis x, slopes up in the direction of the associated wall of the outer cylinder 4 in order to then merge, at the end, into an axially directed channel 62. This channel 62 is formed by an axially oriented, slot-like, radially outwardly opening recess in the lateral surface of the inner cylinder. The channel 62 is covered radially by the associated wall of the outer cylinder 4.

As well as the radial opening 58, which can be seen by way of example in the sectional illustration in FIG. 1, two further radial openings 58′ and 58″ are provided, and these each enclose, as seen in a plane oriented transversely to the axis x, an angle of 90° in relation to this radial opening 58 and, by virtue of the inner-cylinder wall being configured appropriately, are in direct air-flow connection with the grille-wall portion 59.

The axially oriented channel 62 has its end which is directed toward the mouthpiece 6 opening out into an annular chamber 63. The latter forms a vortex chamber. The ceiling 64 of the latter is of cross-sectionally roof-like configuration and is provided with peripherally extending, projecting wings 65, 66. These engage peripherally against the inner wall of the outer cylinder 4 and, as seen in the circumferential direction, leave intermediate spaces 67 between them, through which an air-flow connection is achieved between the annular chamber 63 and a further annular space 68 left between the dispersing-part ceiling portion 51 and the annular-chamber ceiling 64.

The ceiling 64 is secured on the inside wall of the inner cylinder 53 by an axially directed flange 69.

The base of the annular chamber 63 is formed by an annular collar 70 which projects radially outward on the outside wall of the inner cylinder 53 at an axial spacing from the wings 65, 66 of the ceiling 64. It is also the case that this annular collar is supported peripherally on the inside wall of the outer cylinder 4. This annular collar 70 is interrupted by the axially oriented channel 62. The annular chamber 63 is bounded in the radially inward direction by an end-side wall portion which belongs to the inner cylinder 53 and serves for latching the ceiling 64. The resulting annular-chamber wall is provided with slot-like through-passages 71 in order to provide air-flow connection between the annular chamber 63 and the piston-head displacement region 56.

As can furthermore be seen, in particular, from the sectional illustration in FIG. 18, the outer-cylinder wall is provided, level with the annular chamber 63, with two diametrically opposite air-inlet openings 72. These open out tangentially into the annular chamber 63, and this, furthermore, predetermines a common flow direction. Accordingly, a sucking-in action through the air-inlet openings 72 results in a predetermined air flow in the annular chamber 63. The axially oriented channel 62 opens out, as seen in the flow direction, immediately downstream of the mouth of one air-inlet opening 72 in the annular chamber 63, so that the airstream entering into the annular chamber 63 through the axial channel 62 is deflected specifically in the desired vortexing direction via the air-inlet openings 72.

The wings of the ceiling 64, as seen in the circumferential direction, are of different widths. Thus, two diametrically opposite wings 65 are approximately three times the width of the rest of the wings 66, as seen in the circumferential direction. One of these broader wings 65 covers over the mouth region of the axial channel 62 into the annular chamber 63 and, accordingly, forms a deflecting-wall wing 73 for the suction airstream entering into the annular chamber 63 through the axial channel 62.

As can further be seen, in particular, from the illustration in FIG. 27, the wings 66 extend circumferentially, in the exemplary embodiment described, over an angle β of 15°. The intermediate spaces 67 left between the wings 66 and 65 likewise extend circumferentially over an angle α of 15°, while the peripheral edges of the broader wings 65 enclose an angle δ of 45°.

Other distributions are also possible in this respect (for example smaller wings—larger intermediate spaces; larger wings—smaller intermediate spaces; irregular configuration of wings and intermediate spaces).

An interrupter 74 is disposed in the annular chamber 63 adjacent to the mouth of the axial channel 62 in the annular chamber 63, the interrupter being in the airflow direction through the air inlet openings 72. This interrupter limits the circumferential path of the annular chamber 63 and accordingly, as a result of this configuration, this path is of an interrupted form rather than being annular throughout. The rear flank of the interrupter 74, this flank being oriented counter to the flow direction, constitutes a run-on slope 75, connecting the annular-chamber base to the annular-chamber ceiling, which contains the intermediate spaces 67. This causes the airstream in the end region of the annular chamber 63 to be forcibly deflected axially upward into the further annular space 68.

The piston 54, which is retained in a rotationally fixed but axially displaceable manner in the inner cylinder 53, has, in the first instance, a piston head 76 which opens in disk form in the direction of the mouthpiece. This piston head opens conically in cross-section. Two parallel, axially oriented tongues 77 are integrally formed on the underside of the piston disk. The piston 54 consists of a rubber-like material.

Along their lower free periphery, the tongues 77, which accommodate the cross-sectional contour of the guide portion 55 of the inner cylinder 53 on their outside wall, are split in a lip-like manner, and, furthermore, in their free peripheral region, they have material-reinforced sealing surfaces 78.

The flat part of the metering rod 33 is guided between the tongues 77, the sealing surfaces 78, in interaction with the flat part of the metering rod 33, having a stripping and sealing action.

In a basic position of the device according to the illustration in FIG. 1, the free peripheries of the tongues 77, these peripheries being split in a lip-like manner, engage, within an axial depression, against the upper side of the cylinder portion 27.

Furthermore, in this basic position, the disk-like piston head 76 rests in a stop-limited manner on a base region of the piston-head displacement region 56. The encircling peripheral region of the free end of the piston head 76 engages with sealing action against the associated inner wall of the inner cylinder 53.

Furthermore, in this basic position, the head of the metering rod 33, that is to say the radial collar 44 and latching head 45 of the same, rests in the depression created by the disk-like configuration of the piston head 76.

The piston head 76 here is located at an axial distance beneath the ceiling 64.

The device 1 cited functions as follows:

In order to prepare for inhalation, the closure cap 7 is first of all removed by unscrewing. As the closure cap 7 is being unscrewed upwardly, the mentioned coupling results in the outer cylinder 4 being carried along in rotation and, via this outer cylinder, the inner cylinder 53, as well as, in the exemplary embodiment cited, all those parts above the storage-chamber plane which are not connected in a rotationally fixed manner to the housing 3. Accordingly, the metering rod 33 is also carried along in rotation, and furthermore, the action of the closure cap 7 being unscrewed upwardly gives rise, at the same time, to axial displacement of the metering rod 33 via the docking location 41, which causes a helical displacement of the metering chamber 40 into the as yet closed emptying-standby position B according to the illustration in FIGS. 6 and 7, in which it is aligned with the flow channel 60.

By virtue of the metering chamber 40 being disposed eccentrically in relation to the axis of rotation of the metering rod 33, it is filled optimally as a result of penetrating helically through the mass of substance, expediently assisted by the rotor. The larger-diameter opening surface of the metering chamber 40 here is oriented in the direction of rotation.

The simultaneously rotating blade 29 of the rotor R here causes the surrounding mass of substance to be in a constantly loosened state, a shoveling effect being achieved. When the rotor R rotates in the opposite direction—as the closure cap 7 is being screwed on again—the blade 29 interacts with the stator St in order to scrape off substance 2 from the surface of the stator and to press the substance 2 down, as a result of which the mass of substance is evened out. The blade 29 of the rotor R, accordingly, acts on the mass of substance in both directions of rotation.

When the removal-standby position B of the metering rod 33 is reached, the metering rod is secured by means of latching action. For this purpose, the radial collar 44 of the metering rod 33 moves behind latching fingers 79 which are formed on the underside of the ceiling 64.

As the screwing-action displacement of the closure cap 7 continues, the latching in the region of the docking location 41 between the hollow cylinder 43 and the metering rod 33 is eliminated. Accordingly, the noses 47 leave the annular groove 46, whereupon the closure cap 7 can be removed. The device 1 is now prepared for inhalation.

The screwing-action displacement of the closure cap 7 allows for providing sufficient force for producing the latching between the radial collar 44 and latching fingers 79 and, furthermore, for eliminating the latching between the latching head 45 and noses 47 on the cap.

The tongues 77 of the piston 54 cover over the metering chamber 40 on each side. Accordingly, in this position, it is not possible for the sub-quantity 14 of substance to trickle out even in part. Rather, the substance is reliably held captive in the metering chamber 40. This prevents cases of double metering when inhalation is not carried out but the device is closed up again via the closure cap 7. Furthermore, in the removal-standby position B of the metering chamber 40, it is also possible for the device 1 to be put to one side. Even if the device 1 experiences normal kinds of impacts, this does not result in the sub-quantity 14 of substance which is to be inhaled trickling out, which would falsify the inhalation result.

The inhalation operation takes place automatically by the user subjecting the device to suction air, in the simplest case by the user breathing in.

Air is sucked in via the mouthpiece 6, and this, in the first instance, by virtue of the piston head 76 being subjected to the action of air, results in the piston 54 being displaced axially in the direction of the ceiling 64. In the case of the exemplary embodiment illustrated, the pressure required to trigger the device is approximately 2 kgPa. Triggering takes place, as far as possible, in abrupt fashion. The inspiratory effort significantly changes once the piston has begun to move, thus indicating to the user that the inhalation is being performed correctly. Once the piston has assumed its raised position, the flow channel 60 through the metering chamber 40 towards the mouthpiece 6 is no longer blocked by the tongues 77.

In the raised position, the upper free peripheral region of the piston head 76 engages against the underside of an annular wall 80 of the ceiling 64. An audible feedback may be produced by the engagement. The annular space of the inner cylinder 53 which then encloses the free peripheral region of the piston head 76 is widened radially, as a result of which radial flow takes place around the piston 54 in the region of the piston head 76. This results in a main airstream a which flows through the grille-wall portion 59, passing through the radial openings 58, 58′ and 58″, into the piston-head displacement region 56 and passes, by way of the annular-space region left radially outside the piston head 76, through the openings 71 into the annular chamber 63. Approximately 85 to 90% of the total inhalation air volume is transported via this air-flow path.

At the same time, via the always open radial air-inlet openings 72, air is sucked in directly into the annular chamber 63, in order to predetermine the vortexing direction in the annular chamber 63.

By virtue of the axially displaced piston 54, the tongues 77 are likewise displaced axially in order to release the metering chamber 40. The axial displacement of the piston 54 is assisted by the guide portion 55, which accommodates the tongues 77, widening slightly in the direction of the piston head 76, as a result of which there is a reduction in the friction between the tongues 77 and the wall of the guide portion 55. It is also the case that the friction between the tongues 44 and the flat part of the metering rod 33 is minimized to the region of the sealing surfaces 78.

The metering chamber 40 is then located in a removal-release position F, in which it lies freely in the flow path between the flow channel 60 and the intermediate channel portion 61. In the exemplary embodiment as illustrated, approximately 10 to 15% of the inhalation air volume is transported via this substance-transporting airstream b.

The metering chamber is cleared out with through-suction from the flow channel 60, this, furthermore, taking place from the smaller opening surface in the direction of the larger opening surface of the metering chamber 40. The two-fold deflection through approximately 45° in each case into the angled intermediate channel portion 61 and, from the latter, into the axially oriented channel 62 results, in the manner of a baffle-plate effect, in the initial breaking-up of relatively large particles of powder, which further leads to an improved inhalation result.

The substance-laden airstream flowing axially, at relatively high speed, into the annular chamber 63 via the channel 62 is deflected via the deflecting-wall wing 73 and, assisted by the initial flow by way of the radial air-inlet openings 72, in the circumferential direction. Relatively large particles of powder are further broken up on this deflecting-wall wing 73.

As a result of this configuration, the substance-laden airstream is guided outside the piston region. The piston 54 merely has powder-free air flowing around it.

Optimum distribution of the sub-quantity 14 of substance which is to be inhaled is achieved in the annular chamber 63. The substance-laden air passes out through the intermediate spaces 67 for inhalation. Relatively heavy particles of powder which have possibly not been broken up or have not been sufficiently broken up are directed into the annular space 68 at the latest via the interrupter 74.

In the annular chamber 63, the initially substantially axially inflowing airstreams a and b are directed in a common horizontal direction of circulation in order to then pass jointly into the mouthpiece 6, with axial passage through the ceiling 64.

A number of features for indicating successful inhalation to the user are provided. In the first instance, a visual check can be carried out in that the piston 54, once raised by suction air, is retained in its raised position on account of the, albeit low, frictional forces present. The piston 54, or the tongues 77 thereof, can be seen in the removal-standby position B through the radially outwardly open flow channel 60. This can be further assisted by colouring the tongues 77 conspicuous, e.g. in green. Once inhalation has taken place and the piston 54 has been raised correspondingly, the tongues 77 can no longer be seen. Rather, there is a free view of the empty metering chamber 40. Also, the action of the piston 54 striking against the underside of the sealing 64 can be sensed both acoustically and by touch.

Once inhalation has taken place, and alternatively also if it is not desired to effect inhalation from the removal-standby position B, the closure cap 7 is screwed on again, the latching between the radial collar 44 and the latching fingers 79 being eliminated in the first instance by virtue of the latching head 45 being acted upon by the noses 47. The retaining forces of this latching connection are of a correspondingly smaller magnitude than the amount of force which is necessary for deflecting the noses 47. As the action of screwing the closure cap 7 downward continues, the radial collar 44 on the metering rod displaces the piston 54 back again into its basic position. At the same time, with axial displacement and corresponding rotary movement, the metering rod 33 is displaced downward into the storage chamber. The action of the piston 54 being displaced back via the metering rod 33 terminates with the free ends of the tongues 77, which are formed in the manner of lips, striking against the facing ceiling surface of the cylinder part 27. As the downward-screwing displacement continues, the noses 47 finally enter into the annular groove 46 of the metering rod 33. This final latching action is discernable to the user acoustically and by touch, in order to indicate that the closing operation is completed. It is thus also ensured that a latching action between the metering rod 33 and closure cap 7 which causes the metering rod 33, and thus the metering chamber 40, to be carried along into the removal-standby position B is only achieved in the lowermost position of the metering rod 33, in which position the metering chamber 40 is filled. Accordingly, there is always a filled metering chamber 40 available when the metering rod 33 is raised.

Incorrect operation is reliably avoided. Improper closure of the device 1 means that, during the next attempt at inhalation, the metering rod 33, which accordingly has not been raised, on the one hand closes the passage between the flow channel 60 and the intermediate channel portion 61 by way of its flat-part portion. On the other hand, the metering rod 33 continues acting on the associated surface of the piston head 76 via the radial collar 44. Accordingly, when an attempt is made at inhalation, the closure of the flow channel 60 and the blocking of the piston 54 renders it impossible to build up any air flow (with the exception of the small amount of flow via the small radial air-inlet openings 72). This clearly signals incorrect positioning to the user. This can only be eliminated by the device 1 being properly closed.

FIG. 28 shows a schematic view of a part of the metering device of the previous figures in the closed state, where the closure cap is attached to the housing. In FIG. 28, the closure cap 7 is connected to the housing 3. As is apparent from FIG. 28, the closure cap 7 is provided with one or more first rotational stop features 81. The housing 3 is provided with one or more second rotational stop features 82. The first stop features 81 provide first stop faces 83, and the second stop features 82 provide second stop faces 84. The stop faces face in the angular direction. In the closed state of the metering device, the stop faces 84 and 83 abut. The stop faces extend in the axial direction whereas they are oriented in the angular direction such that, when the closure cap 7 is being attached to the housing 3, the stop faces are rotated towards each other, particularly near the end of the connection movement of connecting the closure cap 7 to the housing 3. When the stop faces 84 and 83 abut, the user can be sure that the correct end position of the closure cap has been reached. Likewise, the stop features 81 and 82 are visible from the outside so that a user can check whether the stop features are arranged correctly and whether the stop faces abut in order to confirm whether the closure cap has been applied correctly. The respective stop features may be disposed circumferentially along the housing or the closure cap, respectively. The respective stop feature may be embodied as a tooth having a steep side, which forms a stop face, and a less steep side. Steep sides and less steep sides may be disposed in an alternating manner along the circumference of the cap or the housing, respectively. The slope of the less steep side may be determined by the thread by means of which the closure cap and the housing are connected.

A further advantageous embodiment of the invention is an assembly of an inhaler comprising
a storage chamber, in which a pharmaceutical composition comprises lactose monohydrate having particle size distribution of D10=60-115 micrometer, D50=135-200, D90 is bigger than 220 micrometer and salmeterol xinafoate having particle distribution of D10 less than 2 micrometer, D50 less than 3-6 micrometer, D90 less than 20 micrometer, D99 less than 50 micrometer and fluticasone propionate having particle distribution of D10 less than 1.3 micrometer, D50 less than 4.0 micrometer, D90 less than 10 micrometer, D99 less than 50 micrometer and optionally made by the process of the invention is arranged;
a mouthpiece;
a metering rod provided with a metering chamber, the metering chamber being movable relative to the storage chamber to retrieve a sub-quantity of the pharmaceutical composition—comprises lactose monohydrate having particle size distribution of D10=60-115 micrometer, D50=135-200 micrometer, D90 is bigger than 220 micrometer and salmeterol xinafoate having particle distribution of D10 less than 2 micrometer, D50 less than 3-6 micrometer, D90 less than 20 micrometer, D99 less than 50 micrometer and fluticasone propionate having particle distribution of D10 less than 1.3 micrometer, D50 less than 4.0 micrometer, D90 less than 10 micrometer, D99 less than 50 micrometer
from the storage chamber and to move the sub-quantity into a transfer location outside the storage chamber, wherein, in the transfer location, the sub-quantity is arranged to be retrieved from the metering chamber and to be moved to the mouthpiece by a suction airstream which is generatable by a user suction action which is performed for an inhalation, the suction airstream flowing through a flow channel which is establishable between an air inlet of the assembly and the mouthpiece;
a piston which is movable relative to the metering rod between a blocking position, in which the flow channel is blocked by the piston, and a non-blocking position, in which air is permitted to flow from the air inlet to the mouthpiece via the flow channel, the movement of the piston being actuatable by the user suction action.

According to one embodiment, herein, particularly with respect to the composition, the assembly or the inhaler, the term “D50 less than 3-6 micrometer” means that the D50 value is less than 6 micrometer, or less than 3 micrometer. In another embodiment, the term “D50 less than 3-6 micrometer” means that the D50 value is between 3 and 6 micrometer.

### Example

Firstly 800 g of lactose placed into the blender/Cyclomix Hosokawa equipment/after that 18 g salmeterol xinafoate is added above the lactose layer. Further 800 g of lactose are added into the blender (sandwich layering). The mixture is blended and the resulting blend is discharged and it is stored in a stainless steel container (pre-blend 1).

As a next step 175 g of lactose is filled in an empty blender and 30 g fluticasonpropionate is added onto the lactose layer. Further 175 g of lactose is added into the blender (sandwich layer).

The layers are blended and the resulting blend is discharged and it is stored in a stainless steel container (pre-blend 2).

Next a half of the pre-blend 1 is placed in a blender and pre-blend 2 is added to the pre-blend 1 and the second half of pre-blend 1 is placed to the layers (sandwich layer system) in the blender. Layers are blended together. The resulted blend is sieved and filled—using a volumetric screw—into the storage chamber or reservoir of a dry powder inhaler, preferably further comprising:
a mouthpiece;
a metering rod provided with a metering chamber, the metering chamber being movable relative to the storage chamber to retrieve a sub-quantity of the pharmaceutical composition from the storage chamber and to move the sub-quantity into a transfer location outside the storage chamber, wherein, in the transfer location, the sub-quantity is arranged to be retrieved from the metering chamber and to be moved to the mouthpiece by a suction airstream which is generatable by a user suction action which is performed for an inhalation, the suction airstream flowing through a flow channel which is establishable between an air inlet of the assembly and the mouthpiece; —a piston which is movable relative to the metering rod between a blocking position, in which the flow channel is blocked by the piston, and a non-blocking position, in which air is permitted to flow from the air inlet to the mouthpiece via the flow channel, the movement of the piston being actuatable by the user suction action.

Approximately 1800 units may be filled into the devices using volumetric screw. The specifications of the used APIs and the carrier are as follow:

Lactose monohydrate Ph.Eur PSD: D10: 60-115 μm, D50=135-2 00 μm, D90 is bigger than 220 μm, Salmeterol xinafoate Ph.Eur PSD: D10<2 μm, D50: 3-6 μm, D90<20 μm, D99<50 μm Fluticason propionate Ph.Eur PSD: D10<1.3 μm, D50<4.0 μm, D90<10 μm, D99<50 μm.

### REFERENCE NUMERALS

1 Device
2 Substance
3 Housing
4 Outer cylinder
5 Radial step
6 Mouthpiece
7 Closure cap
8 Internal thread
9 External thread
10 Ribs
11 Grooves
12 Annular shoulder
13 Actuating handle
14 Sub-quantity of substance
15 Storage chamber
16 Pressure-exerting base
17 Compression spring
18 Base cap
19 Latching collar
20 Inner shoulder
21 Hollow piston
22 Annular lip
23 Upright stub
24 Spring chamber
25 Drying-agent capsule
26 Chamber ceiling
27 Cylinder portion
28 Rotary part
29 Blade
30 Rotor ring
31 Sealing bushing
32 Guide opening
33 Metering rod
34 Housing portion
35 Annular seal
36 Radial extension arm
37 Indicating protrusion
38 Viewing window
39 Filling-level indicator
40 Metering chamber
41 Docking location
42 Closure-cap ceiling
43 Hollow cylinder
44 Radial collar
45 Latching head
46 Annular groove
47 Noses
48 Mouthpiece opening
49 Dispersing part
50 Wall
51 Ceiling portion
52 Drying-agent capsule
53 Inner cylinder
54 Piston
55 Guide portion
56 Piston-head displacement region
57 Region wall
58 Radial opening
58′ Radial opening
58″ Radial opening
59 Grille-wall portion
60 Flow channel
61 Intermediate channel portion
62 Channel
63 Annular chamber
64 Ceiling
65 Wing
66 Wing
67 Intermediate spaces
68 Annular space
69 Flange
70 Annular collar
71 Openings
72 Air-inlet openings
73 Deflecting-wall wing
74 Interrupter
75 Run-on slope
76 Piston head
77 Tongues
78 Sealing surfaces
79 Latching finger
80 Annular wall
81 Stop feature
82 Stop feature
83 Stop face
84 Stop face
x Device axis
B Removal-standby position
F Removal-release position
R Rotor
St Stator
U Transfer location
α Angle of intermediate spaces 67
β Angle of wings 66
δ Angle of wings 65
a Main airstream
b Substance-transporting airstream

1. An inhalable pharmaceutical composition comprising
70 mass %-99.5 mass % of pharmaceutically acceptable inhalable carrier suitable for use in dry powder inhalation formulations; and
0.5 mass %-30 mass % of one or two pharmaceutically active ingredient(s),
wherein the composition is suitable for giving improved feedback to a patient about the inhalation of a metered dose in a range between 5 milligram and 10 milligram, and
wherein the composition is suitable for use in a dry powder inhaler.

2. The pharmaceutical composition according to claim 1 comprising two pharmaceutical ingredients suitable for treatment of asthma or COPD or bronchitis.

3. The pharmaceutical composition according to claim 2 wherein the two pharmaceutically active ingredients are selected from levalbuterol, reproterol, clenbuterol, bambuterol, broxaterol salmeterol xinafoate, fluticason propionate budesonide, salbutamol, beclomethasone, formoterol, terbutalin, salmeterol, tulobuterol, indacaterol, isoprenaline, hexoprenaline, tiotropium, ipatropium oxitropium, fenoterol mometason, ciclesonid and rofleponid.

4. The pharmaceutical composition according to claim 3 comprising salmeterol xinafoate and fluticasone propionate or formoterol and budesonide as pharmaceutically active ingredients.

5. The pharmaceutical composition according to claim 4 comprising
70 mass %-99.5 mass % of pharmaceutically acceptable carrier suitable for use in dry powder inhalation formulation; and
0.1 mass %-12.5 mass % of first pharmaceutically active ingredient and 0.4 mass %-17.5 mass % of second pharmaceutical ingredient.

6. The pharmaceutical composition according to claim 5 comprising
88 mass %-99.2 mass % of pharmaceutically acceptable carrier suitable for use in dry powder inhalation formulations; and
0.3 mass %-5.0 mass % of salmeterol xinafoate and 0.5 mass %-7.0 mass % of fluticasone propionate.

7. The composition according to claim 1 wherein the pharmaceutically acceptable carrier is lactose, glucose, maltose, fructose, sucrose, or dextrose, or a hydrate thereof, or a mixture thereof.

8. The composition according to claim 1 wherein the pharmaceutically acceptable carrier comprises lactose or a hydrate thereof.

9. The composition according to claim 1 comprising
lactose monohydrate having a particle size distribution of D10=60-115 micrometer, D50=135-200 micrometer, and D90 greater than 220 micrometer;
salmeterol xinafoate having a particle distribution of D10 less than 2 micrometer, D50 less than 3-6 micrometer, D90 less than 20 micrometer, and D99 less than 50 micrometer; and
fluticasone propionate having a particle distribution of D10 less than 1.3 micrometer, D50 less than 4.0 micrometer, D90 less than 10 micrometer, and D99 less than 50 micrometer.

10. A method of making an inhalable pharmaceutical composition according to claim 1, the method comprising
(i) blending a first part of the carrier and the first pharmaceutically active ingredient to produce a first mixture;
(ii) blending a second part of the carrier and the second pharmaceutically active ingredient to produce a second mixture;
(iii) blending the first and second mixtures to produce a dry powder mixture; and
(iv) filling a dry powder inhaler with the dry powder mixture.

11. A method of making a pharmaceutical composition according to claim 10, the method comprising
(i) blending a first part of the carrier and salmeterol xinafoate to produce a first mixture;
(ii) blending a second part of the carrier and fluticasone propionate to produce a second mixture;
(iii) blending the first and second mixtures to produce a dry powder mixture; and
(iv) filling the dry powder inhaler with the dry powder mixture.

12. A method for treating asthma, COPD or bronchitis, the method comprising administering to a patient in need thereof a pharmaceutical composition according to claim 1.

13. An assembly for a dry powder inhaler comprising
a storage chamber in which a powdery substance is arranged, the powdery substance comprising a pharmaceutical composition according to claim 1;
a mouthpiece;
a metering rod provided with a metering chamber, the metering chamber being movable relative to the storage chamber to retrieve a sub-quantity of the powdery substance from the storage chamber and to move the sub-quantity into a transfer location outside the storage chamber, wherein in the transfer location the sub-quantity is arranged to be retrieved from the metering chamber and to be moved to the mouthpiece by a suction airstream flowing through a flow channel,
wherein the suction airstream is generated by a user suction action performed by an inhalation, and
wherein the flow channel is between an air inlet of the assembly and the mouthpiece; and
a piston which is movable relative to the metering rod between a blocking position, in which the flow channel is blocked by the piston, and a non-blocking position, in which air is permitted to flow from the air inlet to the mouthpiece via the flow channel, the movement of the piston being actuatable by the user suction action.

14. The assembly for a dry powder inhaler according to claim 13, wherein the pharmaceutical composition comprises
70 mass %-99.5 mass % of lactose monohydrate;
0.1 mass %-12.5 mass % of salmeterol xinafoate; and
0.4 mass %-17.5 mass % of fluticasone propionate.

15. The assembly for a dry powder inhaler according to claim 14, wherein the pharmaceutical composition comprises:
88 mass %-99.2 mass % of lactose monohydrate;
0.3 mass %-5.0 mass % of salmeterol xinafoate; and
0.5 mass %-7.0 mass % of fluticasone propionate.

16. The assembly for a dry powder inhaler according to claim 13, wherein the pharmaceutical composition comprises lactose monohydrate having a particle size distribution of D10=60-115 micrometer, D50=135 −200 micrometer, and D90 greater than 220 micrometer;
salmeterol xinafoate having particle distribution of D10 less than 2 micrometer, D50 less than 3-6 micrometer, D90 less than 20 micrometer, and D99 less than 50 micrometer; and
fluticasone propionate having particle distribution of D10 less than 1.3 micrometer, D50 less than 4.0 micrometer, D90 less than 10 micrometer, and D99 less than 50 micrometer.

17. (canceled)

18. A dry powder inhaler, comprising,
an assembly according to claim 13; and
a pharmaceutical composition containing one or more pharmaceutically active ingredient(s) suitable for the treatment of asthma, COPD or bronchitis,
wherein the inhaler is suitable for providing an improved feedback about the inhalation of a metered dose in a range between 5 milligram and 10 milligram during the treatment of asthma, COPD or bronchitis.

19. The method of claim 12, wherein the composition is produced according to a method comprising
(i) blending a first part of the carrier and the first pharmaceutically active ingredient to produce a first mixture;
(ii) blending a second part of the carrier and the second pharmaceutically active ingredient to produce a second mixture;
(iii) blending the first and second mixtures to produce a dry powder mixture; and
(iv) filling a dry powder inhaler with the dry powder mixture.
